(19) United States
(12) Patent Application Publication
(10) Pub. No.: US 2023/0233695 A1
(43) Pub. Date: Jul. 27, 2023

(54) MITHRAMYCIN A NANOPARTICLES FOR CANCER TREATMENT

(71) Applicant: The Research Foundation for the State University of New York, Albany, NY (US)

(72) Inventors: Juntao Luo, Jamesville, NY (US); Jason Horton, Syracuse, NY (US); Dandan Guo, Syracuse, NY (US); Mei Yun Lin, Philadelphia, PA (US)

(21) Appl. No.: 17/890,125

(22) Filed: Aug. 17, 2022

Related U.S. Application Data
(60) Provisional application No. 63/234,029, filed on Aug. 17, 2021.

(51) Int. Cl. A61K 47/60; A61K 9/51; A61K 47/69; A61P 19/08; A61P 35/00

(52) U.S. Cl. CPC A61K 47/60; A61K 9/51; A61K 47/6907; A61P 19/08; A61P 35/00

(57) ABSTRACT

The present disclosure relates to a nanoparticle compound, including: a hydrophilic polymer, a linker, and a drug, wherein the drug is linked to the hydrophilic polymer by the linker, wherein the drug is mithramycin A or a derivative thereof, and wherein the linker is boronic acid or a boronic acid derivative. For example, the present disclosure includes one or more nanoparticles including one or more conjugates, and methods of using the nanoparticles for drug delivery, and treating a disease such as Ewing sarcoma or osteosarcoma.

### CROSS REFERENCE TO RELATED APPLICATIONS

This application claims the benefit of the filing date of U.S. Provisional Application No. 63/234,029 filed Aug. 17, 2021. The content of this earlier filed application is hereby incorporated by reference herein in its entirety.

### FIELD OF THE INVENTION

The present disclosure is directed to a drug delivery system suitable for treating subjects in need thereof. For example, a nanotechnology drug delivery system of the present disclosure is suitable for delivering a drug or prodrug such as mithramycin a and/or derivatives thereof to a subject in need, such as a subject having cancer. In embodiments, the present disclosure provides a nanoparticle including one or more conjugates of the present disclosure, methods of using the nanoparticles for drug delivery, and treating a disease such as Ewing sarcoma or osteosarcoma.

### BACKGROUND

Ewing sarcoma (ES) is classified as a small, round blue cell tumor, and is the second most common malignant bone tumor in children after osteosarcoma. It is an aggressive osteolytic tumor with poorly differentiated cells and high metastatic potential. The survival rate of patients diagnosed with ES stand at 70% in cases with a solitary primary tumor but drops to less than 25% for patients with recurrent or metastatic disease. One of the unique signatures of ES is that near 85% of ES tumors possess a chromosomal translocation t(11;22)(q24;q12) involving the EWS breakpoint region 1 (EWSR1) gene and the Friend leukemia virus integration site 1 (Fli1) gene. Extensive evidence supports that the tumorigenesis, progression, and survival of ES tumor cells depends on continued activity of EWS:Fli1. This translocation is only present in tumor cells, making it an attractive therapeutic target. However, it is well known that targeting a transcription factor can be extremely difficult and seems “undruggable” since EWS:Fli1 possesses intrinsically disordered regions and it lacks intrinsic enzymatic function so cannot be directly targeted by a conventional, rationally-designed small molecule.

Mithramycin A (MithA) is a DNA binding RNA synthesis inhibitor. It was first tested as a chemotherapeutic agent in the 1960s to treat hypercalcemia, Paget's disease of bone, testicular cancer, chronic myeloid leukemia, and ES. In recent years, MithA was identified in a high-throughput screen of over 50,000 compounds as a potent and specific EWS:Fli1 transcriptional inhibitor. Studies have demonstrated that MithA can inhibit proliferation and radiosensitize EWS:Fli1 expressing cells in vitro and delayed growth of ES xenografts in vivo. MithA can suppress the EWS:Fli1 downstream target NR0B1 in vitro and in vivo in a cell-based luciferase reporter model. Unfortunately, a Phase I/II clinical trial using MithA as single-agent therapy for patients with end-stage ES (NCT01610570) was terminated during dose escalation because 2/8 patients developed asymptomatic reversible grade 4 alanine aminotransferase (ALT)/aspartate aminotransferase (AST) elevation exceeding three time the upper limit of normal, indicating acute hepatic necrosis. In this trial, MithA did not reach serum levels (˜50 nM) hypothesized to effectively inhibit EWS:Fli1 activity before the onset of adverse effects. Another clinical trial that used MithA for lung, esophagus, and other chest cancers (NCT1624090) reported 10/12 treated patients developed hepatotoxicity with ALT and AST elevations greater than 13-fold increase in geometric mean. No intervention was needed for 8 patients which experienced clinically significant (grade 3) transaminase elevations in this trial, which resolved in 1-3 weeks after the dose de-escalation, and rarely led to jaundice. A study following this clinical trial suggested that MithA-induced liver toxicity through dysregulated bile acid disposition since MithA alters expression of hepatic bile acid transporters and MithA inhibits farnesoid X receptor XR expression by functioning as an antagonist of the ligand-dependent activation of FXR.

Despite the promising pre-clinical results of MithA against ES, it is known to cause significant toxicity including thrombocytopenia, elevated liver function tests, anorexia, nausea, vomiting, infusional fever, mucositis, bleeding tendencies, electrolyte abnormalities, proteinuria and elevated BUN/creatinine in some patients. Thus, a less systemically toxic formulation is needed for realization of MithA as a targeted treatment for EWS:Fli1⁺ tumors.

In the past two decades, one of the main advancements in nanotechnology is the development of nanoparticle (NP) drug delivery systems. In tumor tissues, rapid growth of blood vessels results in a disorganized capillary network that exhibits increased permeability to large solutes, relative to normal tissues. This enhanced permeability allows NP to preferentially exit the circulation in the tumor environment, promoting drug concentration and retention in the extravascular space of the tumor. The so-called enhanced permeability and retention (EPR) effect, in combination with a drug-laden nanoparticle can be exploited to achieve higher intratumoral drug concentration while also reducing exposure of normal tissues to the toxic agent.

Prior-art-of-interest includes: U.S. Patent Publication No. 20150056139 relating to telodendrimers and nanocarriers and methods of using the same; U.S. Patent Publication No. 20190112423 relating to reversibly cross-linked micelle systems; WO2010/039496 relating to a nanocarriers having an interior and an exterior, the nanocarriers including at least one conjugate, wherein each conjugate includes polyethylene glycol (PEG) polymer; WO2013/096388 and WO2012/158622 relating to amphiphilic telodendrimers that aggregate to form nanocarriers characterized by hydrophobic core and a hydrophilic exterior (all of which are entirely incorporated herein by reference). However, these publications do not show nanoparticles of the present disclosure.

There is a continuing need for nanoparticles, drug delivery vehicles, and systems that overcome targeting and toxicity deficiencies.

### SUMMARY

The present disclosure provides a nanoparticle, methods of using the nanoparticle for drug delivery, and treating a disease such as Ewing sarcoma or osteosarcoma.

In embodiments, the present disclosure relates to a nanoparticle compound, including: a hydrophilic polymer, a linker, and a drug, wherein the drug is linked to the hydrophilic polymer by the linker, wherein the drug is mithramycin A or a derivative thereof, and wherein the linker is boronic acid or a boronic acid derivative. In embodiments, the hydrophilic polymer is polyethylene glycol (PEG) having a molecular weight of 1-100 kDa. In embodiments, the boronic acid derivative is selected from the group consisting of 3-carboxyphenylboronic acid, 4-carboxyphenylboronic acid, 5-carboxyphenylboronic acid, 3-carboxy-5-nitrophenylboronic acid pinacol ester, and combinations thereof. In embodiments, the compound is characterized as a PEGylated mithramycin A prodrug, or mithramycin A conjugated micelle.

In embodiments, the present disclosure relates to a method of delivering a drug, the method including administering a nanoparticle compound of the present disclosure to a subject in need thereof; and cleaving the is mithramycin A or a derivative thereof in situ, such that the drug is released from the nanoparticle. In embodiments, the subject has a disease characterized as Ewing sarcoma or osteosarcoma.

In embodiments, the present disclosure relates to a method of making the nanoparticle compound of the present disclosure, including contacting phenylboronic acid-containing telodendrimers with mithramycin A under conditions sufficient to form one or more nanoparticles.

In embodiments, the present disclosure includes a compound characterized as PEG^{5k}(5-NO₂m-BOH₂)ₙ-MithA wherein n−1-2.

In embodiments, the present disclosure includes a compound characterized as PEG^{5k}(5-NO₂m-BOH₂)₁-MithA or PEG^{5k}(5-NO₂m-BOH₂)₂.-MithA.

In embodiments, the present disclosure relates to a method of making a PEGylated mithramycin A prodrug including, contacting one or more phenylboronic acid-containing telodendrimers with a mithramycin A under conditions sufficient to link the one or more phenylboronic acid-containing telodendrimers with the mithramycin A.

In embodiments, the present disclosure relates to a method of making a mithramycin A prodrug including, contacting one or more phenylboronic acid-containing telodendrimers with a mithramycin A under conditions sufficient to link the one or more phenylboronic acid-containing telodendrimers with the mithramycin A. In embodiments, the one or more phenylboronic acid-containing telodendrimers comprises one or more hydrophilic polymers.

In embodiments, the present disclosure provides a nano-formulation of MithA that is approximately 150 nm in diameter, conjugated to a polymer (PEK^{5K}NO₂B). In embodiments, the nano-formulation includes a pH-sensitive boronic linkage (MANP) to further enhance intratumoral release of the free drug from the NP by taking advantage of the acidic tumor microenvironment. Data indicates that, in embodiments, MANP enters tumor cells by endocytosis and is trafficked through the endo-lysosomal pathway. In embodiments, drug release was prevented in cells treated with Bafilomycin A, a molecule that prevents endo-lysosomal acidification by inhibiting V-ATPase activity. In embodiments, MANP will preferentially accumulate in the tumor sites, reduce hepatotoxicity while maintaining anti-tumor efficacy relative to the free MithA. The purpose of this study was to test embodiments, of the present disclosure such as MANP in a pre-clinical animal model. In vitro studies demonstrated MANP is excellent at inhibiting the EWS:Fli1⁺ tumor cells. MANP showed a comparable extent of tumor regrowth inhibition as free MithA in EWS:Fli1⁺ xenograft mice, and prolonged the median survival of tumor-bearing mice from 6 days (control) to 14 days. The most remarkable finding is that MANP were both non-toxic and effective in our pre-clinical model. Whereas in free MithA treated mice MithA-induced liver toxicity and thrombocytopenia was observed. Taken together, results demonstrate a promising molecularly targeted treatment using nanodrug delivery system against tumors with EWS:Fli1 oncogenic fusion.

In embodiments, the present disclosure includes a compound comprising the following structure: (PEG^{5k}(5-NO₂m-BOH₂))ₙ-MithA, wherein (n=1-3).

In embodiments, the present disclosure includes a compound comprising the following structure: (PEG^{5k}(5-NO₂m-BOH₂))₁-MithA; (PEG^{5k}(5-NO₂m-BOH₂))₂-MithA; or (PEG^{5k}(5-NO₂m-BOH₂))₃-MithA.

In embodiments, the present disclosure includes a nanoparticle comprising: a compound comprising one or more of the following: (PEG^{5k}(5-NO₂m-BOH₂))₁-MithA; (PEG^{5k}(5-NO₂m-BOH₂))₂-MithA; (PEG^{5k}(5-NO₂m-BOH₂))₃-MithA; or combinations thereof.

The illustrative aspects of the present disclosure are designed to solve the problems herein described and/or other problems not discussed.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Embodiments of the present disclosure, briefly summarized above and discussed in greater detail below, can be understood by reference to the illustrative embodiments of the disclosure depicted in the appended drawings. However, the appended drawings illustrate only typical embodiments of the disclosure and are therefore not to be considered limiting of scope, for the disclosure may admit to other equally effective embodiments.

FIG. 1 depicts a schematic illustration of in vivo anti-tumor efficacy study. in accordance with the present disclosure.

FIG. 2A-2I depict dose-response curves for MithA and MANP measured by XTT assay in accordance with the present disclosure.

FIGS. 3A-3C depict MithA and MANP down-regulated RAD51 and NR0B1.

FIGS. 4A-4E depict quantification of Rad51 and NR0B1 mRNA expression by Rt-qPCR.

FIGS. 5A-5D depict tumor targeting and Tissue Biodistribution of Cy5.5-labeled MANP in TC-71 (EWS:Fli1⁺) Xenograft Tumor-Bearing Nude Mice in accordance with the present disclosure.

FIGS. 6A and 6B depict anti-tumor activity of MithA and MANP in TC-71 (EWS:Fli1+) xenograft tumor-bearing nude mice. FIGS. 6C and 6D depict mean tumor volume±standard deviation and body weight change (%), respectively.

FIGS. 7A-7R depict MithA Liver Toxicity Profile in Female C57BL/6J Mice.

FIGS. 8A-8T depict MithA Hematology Profile in Female C57BL/6J Mice.

FIGS. 9A-9Q depict data for blood chemistry for single intravenous injection of 4 mg/kg MithA MANP in Female c57BL/6J Mice.

FIGS. 10A-10C depict Representative formalin-fixed paraffin sections (5 μm) of liver tissue from mice treated with five doses of saline Vehicle, BlankNP, Free MithA (2 mg/kg) or MANP (2 mg/kg eq) given on alternating days.

FIGS. 11A-11Q depict MANP Alleviated MithA-induced Liver Toxicity in Female C57BL/6J Mice.

FIGS. 12A-12L depict MANP Alleviated MithA-induced Platelet Dysfunction.

FIG. 13 depicts a synthesis route of PEG^{5k}(m-BOH₂)₁, PEG^{5k}(o-BOH₂)₁, PEG^{5k}(p-BOH₂)₁, and PEG^{5k}(5-NO₂m-BOH₂)₁ in accordance with the present disclosure.

FIG. 14 depicts synthesis route of PEG^{5k}(m-BOH₂)₂, PEG^{5k}(o-BOH₂)₂, PEG^{5k}(p-BOH₂)₂, and PEG^{5k}(5-NO₂m-BOH₂)₂ in accordance with the present disclosure.

FIG. 15 depicts the preparation of PEGylated Mithramycin A prodrug in accordance with an embodiment of the present disclosure.

FIG. 16 depicts TLC of free mithramycin A, PEG^{5k}(5-NO₂m-BOH₂)₁, and PEG^{5k}(5-NO₂m-BOH₂)₁-MithA (3:1 molar ratio) and prodrug after 30 min incubation at pH 7.4 and 5.5.

FIGS. 17A and 17B depict fluorescence of free mithramycin A and PEG^{5k}(5-NO₂m-BOH₂)₁-MithA at pH 7.4. and particle size of PEG^{5k}(5-NO₂m-BOH₂)₁-MithA measured by DLS particle sizer, respectively.

It is noted that the drawings of the disclosure are not necessarily to scale. The drawings are intended to depict only typical aspects of the disclosure, and therefore should not be considered as limiting the scope of the disclosure. In the drawings, like numbering represents like elements between the drawings.

### DETAILED DESCRIPTION

The present disclosure is directed to a drug delivery system suitable for treating subjects in need thereof. For example, a nanotechnology drug delivery system of the present disclosure is suitable for delivering a drug or prodrug such as mithramycin and/or derivatives thereof to a subject in need, such as a subject having cancer. The present disclosure provides a nanoparticle including one or more conjugates of the present disclosure, and methods of using the nanoparticles for drug delivery, and/or treating a disease such as Ewing sarcoma or osteosarcoma.

The present disclosure provides a nanoparticle including one or more conjugates of the present disclosure, and methods of using the nanoparticles for drug delivery, treating a disease such as Ewing sarcoma or osteosarcoma.

In embodiments, the present disclosure relates to a nanoparticle compound, including: a hydrophilic polymer, a linker, and a drug, wherein the drug is linked to the hydrophilic polymer by the linker, wherein the drug is mithramycin A or a derivative thereof, and wherein the linker is boronic acid or a boronic acid derivative. In embodiments, the hydrophilic polymer is polyethylene glycol (PEG) having a molecular weight of 1-100 kDa. In embodiments, the boronic acid derivative is selected from the group consisting of 3-carboxyphenylboronic acid, 4-carboxyphenylboronic acid, 5-carboxyphenylboronic acid, 3-carboxy-5-nitrophenylboronic acid pinacol ester, and combinations thereof. In embodiments, the compound is characterized as a PEGylated mithramycin A prodrug, or mithramycin A conjugated micelle.

In embodiments, the present disclosure relates to a method of delivering a drug, the method including administering a nanoparticle compound of the present disclosure to a subject in need thereof; and cleaving the is mithramycin A or a derivative thereof in situ, such that the drug is released from the nanoparticle. In embodiments, the subject has a disease characterized as Ewing sarcoma or osteosarcoma.

In embodiments, the present disclosure relates to a method of making the nanoparticle compound of the present disclosure, including contacting phenylboronic acid-containing telodendrimers with mithramycin A under conditions sufficient to form one or more nanoparticle.

In embodiments, the present disclosure includes a compound characterized as PEG^{5k}(5-NO₂m-BOH₂)ₙ-MithA wherein n−1-2.

In embodiments, the present disclosure includes a compound characterized as PEG^{5k}(5-NO₂m-BOH₂)₁-MithA or PEG^{5k}(5-NO₂m-BOH₂)₂.-MithA.

In embodiments, the present disclosure relates to a method of making a PEGylated mithramycin A prodrug including, contacting one or more phenylboronic acid-containing telodendrimers with a mithramycin A under conditions sufficient to link the one or more phenylboronic acid-containing telodendrimers with the mithramycin A.

In embodiments, the present disclosure relates to a method of making a mithramycin A prodrug including, contacting one or more phenylboronic acid-containing telodendrimers with a mithramycin A under conditions sufficient to link the one or more phenylboronic acid-containing telodendrimers with the mithramycin A. In embodiments, the one or more phenylboronic acid-containing telodendrimers comprises one or more hydrophilic polymers.

In embodiments, the present disclosure relates to tuning a prodrug to a predetermined rate of release in situ. In embodiments, various prodrug molecules include one or more predetermined linkers of the present disclosure.

### Definitions

As used in the present specification, the following words and phrases are generally intended to have the meanings as set forth below, except to the extent that the context in which they are used indicates otherwise.

As used herein, the singular forms “a”, “an”, and “the” include plural references unless the context clearly dictates otherwise. Thus, for example, references to “a compound” include the use of one or more compound(s).

As used herein the terms “about,” “approximately,” and the like, when used in connection with a numerical variable, generally refers to the value of the variable and to all values of the variable that are within the experimental error (e.g., within the 95% confidence interval [CI 95%] for the mean) or within ±10% of the indicated value, whichever is greater.

As used herein the terms “drug,” “drug substance,” “active pharmaceutical ingredient,” and the like, refer to a compound that may be used for treating a subject in need of treatment.

As used herein the term “excipient” or “adjuvant” refers to any inert substance.

As used herein the terms “drug product,” “pharmaceutical dosage form,” “dosage form,” “final dosage form” and the like, refer to a pharmaceutical composition that is administered to a subject in need of treatment and generally may be in the form of inhalers, tablets, capsules, sachets containing powder or granules, liquid solutions or suspensions, patches, and the like.

As used herein the term “pharmaceutically acceptable” substances refers to those substances, which are within the scope of sound medical judgment suitable for use in contact with the tissues of subjects without undue toxicity, irritation, allergic response, and the like, and effective for their intended use.

As used herein the term “pharmaceutical composition” refers to the combination of one or more drug substances, one or more excipients, and one or more pharmaceutically acceptable vehicles with which the one or more drugs is administered to a subject.

As used herein, the term “pharmaceutically acceptable salt” refers to a salt of a compound, which possesses the desired pharmacological activity of the parent compound. Non-limiting examples of pharmaceutically acceptable salts include: acid addition salts, formed with inorganic acids such as hydrochloric acid, hydrobromic acid, sulfuric acid, nitric acid, phosphoric acid, and the like; or formed with organic acids; and salts formed when an acidic proton present in the parent compound is replaced by a metal ion, for example, an alkali metal ion, an alkaline earth ion, or an aluminum ion. In embodiments, MithA and MithA conjugates of the present disclosure may be in a pharmaceutically acceptable salt form.

As used herein the term “pharmaceutically acceptable vehicle” refers to a diluent, adjuvant, excipient or carrier with which a compound is administered.

As used herein the term “subject” includes humans, animals or mammals. The terms “subject” and “patient” may be used interchangeably herein.

As used herein the term “therapeutically effective amount” means the amount of a compound that, when administered to a subject for treating or preventing disease is sufficient to effect such treatment or prevention of the disease and related symptoms. A “therapeutically effective amount” can vary depending, for example, on the compound, the severity of the disease, the etiology of the disease, the age of the subject to be treated, comorbidities of the subject to be treated, existing health conditions of the subject, and/or the weight of the subject to be treated. A “therapeutically effective amount” is an amount sufficient to alter the subjects' natural state.

As used herein the term “prevent”, “preventing” and “prevention” of disease means (1) reducing the risk of a patient who is not experiencing symptoms of disease from developing a disease, or (2) reducing the frequency of, the severity of, or a complete elimination of disease already being experienced by a subject.

As used herein the term “prodrug” refers to a biologically inactive compound which can be metabolized or altered in the body to produce a drug.

As used herein the term “treat”, “treating” and “treatment” of disease means reducing the frequency of symptoms of disease, eliminating the symptoms of disease, avoiding or arresting the development of symptoms of disease, ameliorating or curing an existing or undesirable symptom caused by disease, and/or reducing the severity of symptoms of a disease such as cancer or sarcoma.

Before embodiments are further described, it is to be understood that this disclosure is not limited to particular embodiments described, as such may, of course, vary. It is also to be understood that the terminology used herein is for the purpose of describing particular embodiments only and is not intended to be limiting.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are entirely incorporated herein by reference to disclose and describe the methods and/or materials in connection with which the publications are cited.

### Description of Certain Embodiments

The present disclosure is directed to a drug delivery system suitable for treating subjects in need thereof. For example, a nanotechnology drug delivery system of the present disclosure is suitable for delivering a drug or prodrug such as mithramycin and/or derivatives thereof to a subject in need, such as a subject having cancer. In embodiments, the present disclosure provides a nanoparticle including one or more conjugates of the present disclosure, and methods of using the nanoparticles for drug delivery, and treating a disease such as Ewing sarcoma or osteosarcoma. In embodiments, the nanoparticles are characterized as pharmaceutically acceptable, and may be administered to a subject in need thereof if a therapeutically effective amount.

In some embodiments, the present disclosure is directed to a drug delivery system suitable for treating subjects in need thereof. For example, a nanotechnology drug delivery system of the present disclosure is suitable for delivering a drug or prodrug such as mithramycin a and/or derivatives thereof to a subject in need, such as a subject having cancer. In some embodiments, mithramycin a refers to (2S,3S)-2-[(2S,4R,5R,6R)-4-[(2S,4R,5S,6R)-4-[(2S,4S,5R,6R)-4,5-dihydroxy-4,6-dimethyloxan-2-yl]oxy-5-hydroxy-6-methyloxan-2-yl]oxy-5-hydroxy-6-methyloxan-2-yl]oxy-3-[(1 S,3S,4R)-3,4-dihydroxy-1-methoxy-2-oxopentyl]-6-[(2S,4R,5R,6R)-4-[(2S,4R,5S,6R)-4,5-dihydroxy-6-methyloxan-2-yl]oxy-5-hydroxy-6-methyloxan-2-yl]oxy-8,9-dihydroxy-7-methyl-3,4-dihydro-2H-anthracen-1-one, or a pharmaceutically acceptable salt thereof.

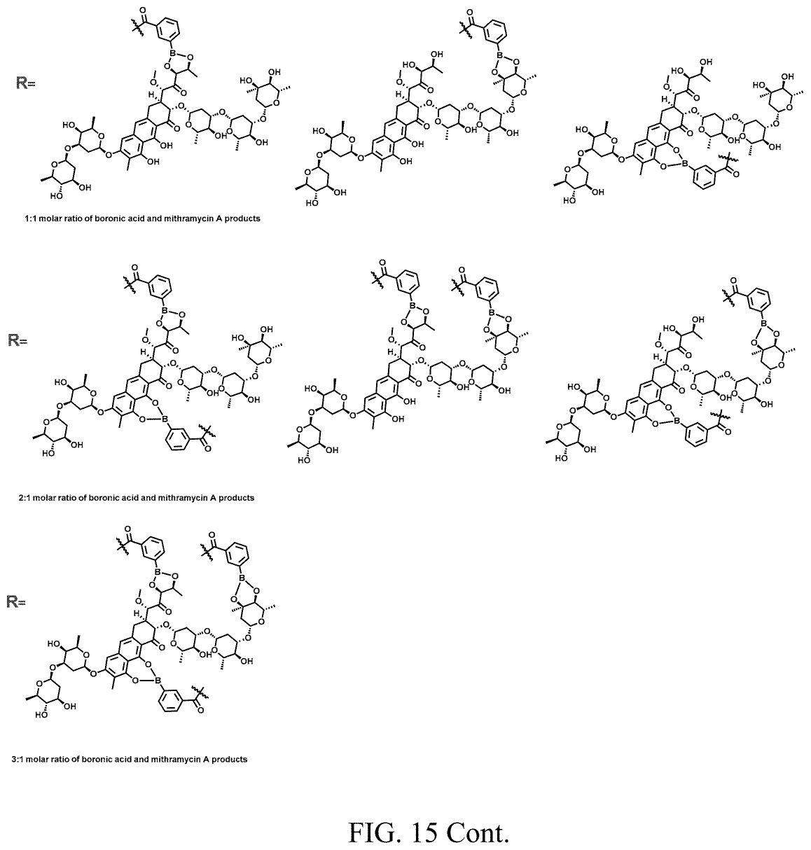

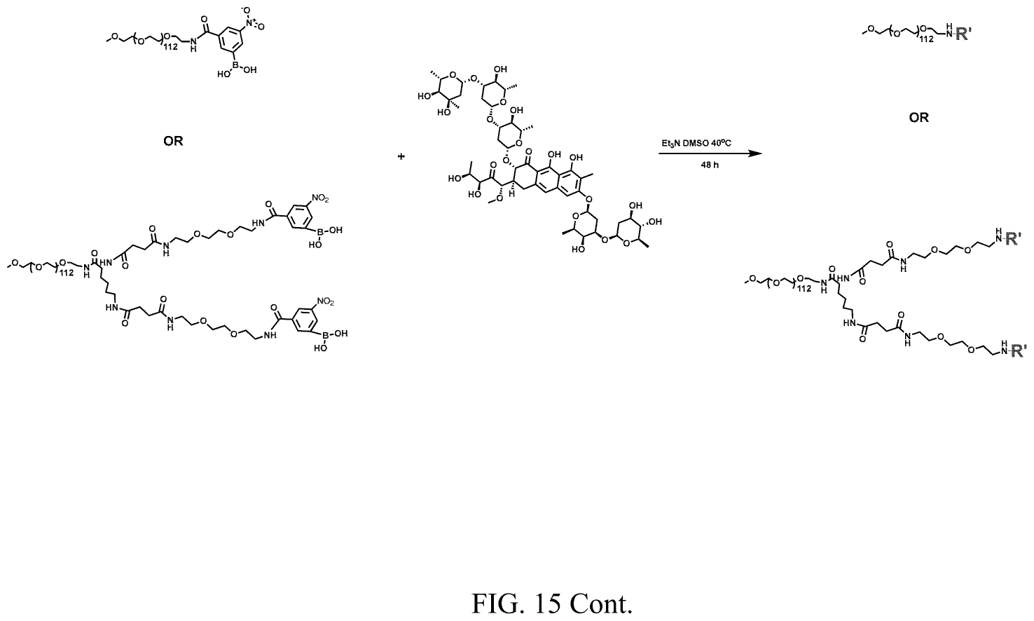

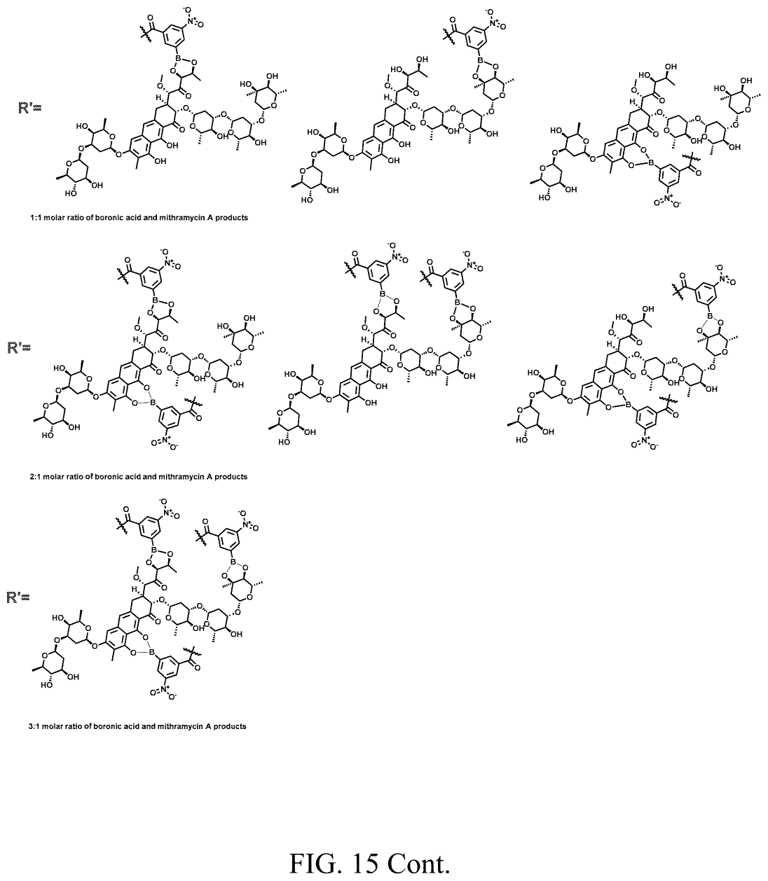

In some embodiments, the present disclosure provides a nanoparticle including one or more conjugates of the present disclosure, and methods of using the nanoparticles for drug delivery, treating a disease such as Ewing sarcoma or osteosarcoma. In embodiments, the nanoparticles comprising one or more of the compositions characterized as R and R′ as depicted in FIG. 15. Non-limiting examples of nanoparticles of the present disclosure includes PEGylated Mithramycin A prodrug depicted in FIG. 15. However, materials other than PEG may be suitable for use herein. In some embodiments, the nanoparticle is further characterized as a particulate carrier, a nanocapsule (NC) or a nanosphere (NS), which is biocompatible and sufficiently resistant to chemical and/or physical destruction, such that a sufficient amount of the nanoparticles remain substantially intact after administration into a human or animal body and for sufficient time to be able to reach the desired target tissue or organ. In embodiments, nanoparticles are spherical in shape, having an average diameter of up to 500 nanometers. Where the shape of the particle is not spherical, the diameter refers to the longest dimension of the particle. In embodiments, the compound, such as R or R′ is preselected to remain substantially intact after administration into a human or animal body and for sufficient time to be able to reach the desired target tissue or organ and react with the low pH environment of a tumor to cleave the drug from the nanoparticle.

In embodiments, the present disclosure includes a compound comprising the following structure: (PEG^{5k}(5-NO₂m-BOH₂))ₙ-MithA, wherein (n=1-3).

In embodiments, the present disclosure includes a compound comprising the following structure: (PEG^{5k}(5-NO₂m-BOH₂))₁-MithA; (PEG^{5k}(5-NO₂m-BOH₂))₂-MithA; or (PEG^{5k}(5-NO₂m-BOH₂))₃-MithA.

In embodiments, the present disclosure includes A nanoparticle comprising: a compound comprising one or more of the following: (PEG^{5k}(5-NO₂m-BOH₂))₁-MithA; (PEG^{5k}(5-NO₂m-BOH₂))₂-MithA; (PEG^{5k}(5-NO₂m-BOH₂))₃-MithA; or combinations thereof.

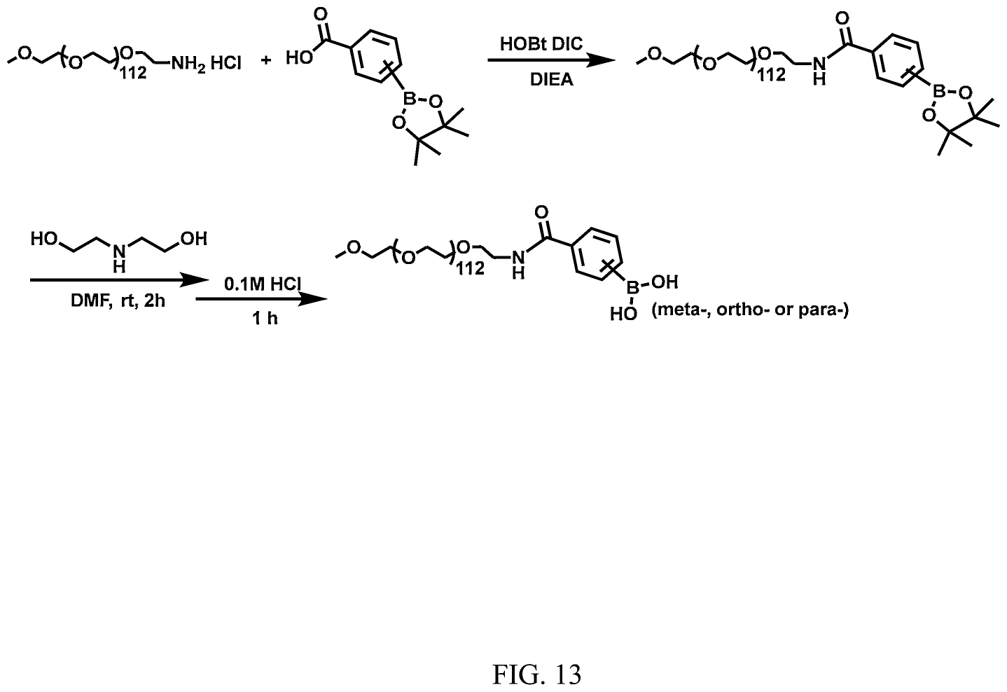

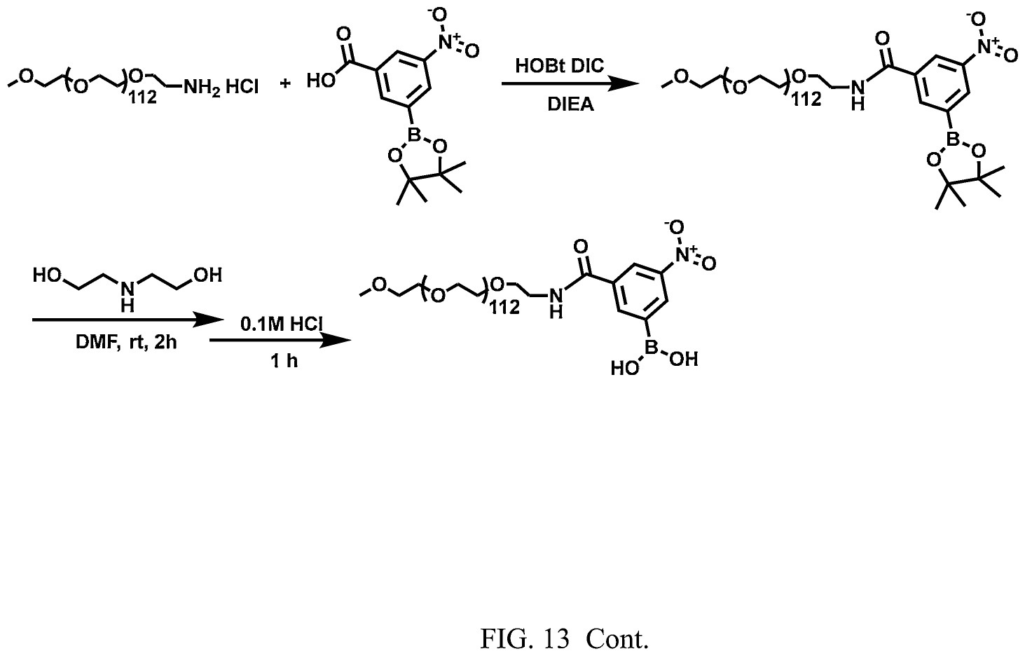

In some embodiments, the present disclosure provides a method of making nanoparticle including one or more conjugates of the present disclosure. Non-limiting examples of making the nanoparticles of the present disclosure are described in Example 2 below and depicted in FIGS. 13-15.

The present disclosure also provides one or more pharmaceutical compositions including the nanoparticle(s) of the present disclosure. The compositions of the present disclosure can be prepared in a wide variety of oral, parenteral and topical dosage forms. In embodiments, suitable pharmaceutical dosage forms include those described in in section “V” of WO2021/126970 at paragraphs [0083] through [0118] (herein incorporated by reference). It is noted that WO2021/126970 is entirely incorporated by reference herein.

### EXAMPLES

Summary of Example I: Ewing sarcoma (ES) is an aggressive pediatric musculoskeletal malignancy. Regrettably, neither the approach to treating ES, nor the survival statistics have improved in the past 30 years. Mithramycin A is a DNA binding RNA synthesis inhibitor, and pre-clinical studies from our lab and others have demonstrated that MithA is an effective inhibitor of the EWS:Fli1 oncogene that drives approximately 85% of ES cases. In recent years, researchers conducted a phase 1/11 trial to determine the effectiveness and toxicities of MithA in children and adults with refractory EWS:Fli1⁺ EWS. Unfortunately, the study was stopped during dose escalation due to reversible grade 4 hepatoxicity in 2 out of 8 patients treated. Hence failed to safely administrate MithA while preserving its anti-tumor efficacy. A novel nano-formulation of MithA that is conjugated to PEG^{5K}NO₂B polymer (MANP) is provided herein. The purpose of this study was to test the MANP formulation of the present disclosure in cell culture and pre-clinical animal models. Here, the MANP mitigates MithA-induced liver toxicity and thrombocytopenia while maintaining anti-tumor efficacy. The most remarkable result to emerge from this study was that the MANP formulation of the present disclosure can prolong the survival of EWS:Fli1 tumor-bearing mice from a median survival of 6 days in vehicle and blank polymer groups to 14 days, and mice did not observe any sign of liver toxicity and thrombocytopenia as the free MithA group did. The data indicate that MANP improves the therapeutic index of MithA for treatment of ES expressing the EWS:Fli1⁺ fusion.

### Methods and Materials

Cell culture and animals. Cell lines TC-71 and CHLA-25 were obtained from the Childhood Cancer Repository of the Children's Oncology Group (Texas Tech University Health Sciences Center, Lubbock, Tex.). Cell lines RD-ES (HTB-166), SK-ES-1 (HTB-86), A673 (CRL-1598), 293T (CRL-11268), THP-1 (TIB-202), and MEG-01 (CRL-2021) were obtained from American Type Culture Collection (ATCC, Manassas, Va.). Hep-G2 cells were obtained from Dr. Jay Henderson's lab at Syracuse University. All cells were cultivated in the growth media formulated as described in Table 1 and were maintained at 37° C. in a humidified 5% CO2 incubator.

TABLE 1

| | Media component | | | | |
|---|---|---|---|---|---|
| | | | Antibiotic/Anti- | | |
| | | | mycotic solution, | Glutamax, | |
| | | Fetal Calf Serum, Atlania Biologicals | Life Technologies | Life Technologies | |
| | Base Media | [symbol] 11150 Lot L14142 | #15240-062 | #35050-061 | Other supplements |
| A673 | DMEM, Corning #10-014-CV | 10% | 1% | 1% | Sodium Py [symbol] Sigma # [symbol] 8636 |
| RD-ES | RPMI-1640, ATCC #30-2001 | 15% | 1% | 1% | — |
| SK-ES-1 | McCoy's 5A, ATCC #30-2007 | 15% | 1% | 1% | — |
| CHLA-25 | IMDM, ATCC #30-2005 | 20% | 1% | 1% | 1% ITS+, Sigma #12521 |
| TC-71 | IMDM, ATCC #30-2005 | 20% | 1% | 1% | 1% ITS+, Sigma #12521 |
| HEK293 [symbol] | DMEM, Corning #10-017-CV | 10% | 1% | 1% | Sodium Py [symbol] Sigma # [symbol] 8636 |
| HepG2 | EMEM, ATCC #30-2003 | 10% | 1% | 1% | — |
| MEG-01 | RPMI-1640, ATCC #30-2001 | 10% | 1% | 1% | — |
| THP-1 | RPMI-1640, ATCC #30-2001 | 10% | 1% | 1% | 2- [symbol] (0.05 mM final), |
| | | | | | Sigma#60242 |

[symbol]

indicates data missing or illegible when filed

Female C57BL/6J mice (strain stock #000664) aged 6-8 weeks and female outbred athymic FoxN1^{nu/nu} homozygous nude mice (strain Nu/J stock #002019) aged 8 weeks were purchased from The Jackson Laboratory (Bar Harbor, Me.). All the animals were housed in pathogen-free conditions and were allowed to acclimatize to the local environment for at least 7 days before starting experiments. All animal experiments were performed in compliance with protocol approved by the Institutional Animal Care and Use Committee of Upstate Medical University and were conducted in accordance with the US Public Health Service Policy on Humane Care and Use of Laboratory Animals (PHS Assurance A3514-01).

Cell viability assays. For viability assays, four EWS:Fli1 fusion tumor cell lines (RD-ES, SK-ES-1, A673, and TC-71), one human embryonic kidney cell line (293.T), one ES tumor cell line with ERG fusion (CHLA-25), one fetal kidney cell line (HEK293.T), one hepatoblastoma cell line (HepG2), one chronic myeloid leukemia (MEG-01), and one acute monocytic leukemia (THP-1) cell line were plated at 5000 cells/well in 100 μL growth media in 96-well plates. After overnight incubation, 50 μL of growth media containing mithramycin A (MithA) (Cayman Chemical, Ann Arbor, Mi. Item Number 11434) or boronic acid-conjugated nano-MithA (MANP) (0.12 nM to 500 nM, final) or dimethyl sulfoxide (DMSO, 0.01% v/v) vehicle was added to the wells, and cell viability assays were performed 72 hours later. The cells were incubated with the activated-XTT solution (mixture of 2% final concentration of activation reagent with the XTT reagent) from ATCC (Catalog Number 30-1011K, XTT Cell Proliferation Assay Kit) for 3 hours prior to recording absorbance. The cell viability was determined by measuring the absorbance wavelength at 475 nm and reference wavelength at 650 nm on an Infinite M200 plate reader (Tecan Group, Ltd. Mannedorf, Switzerland). Raw data for all assays were corrected for background absorbance and normalized to the 0.01% DMSO vehicle control to construct dose-response curves and determine IC₅₀ values. All dose-response viability assays were independently replicated at least three times with six technical replicate wells.

In vivo biodistribution and clearance of Cy5.5-MANP. Xenograft tumors of TC-71 cells were established in homozygous Female FoxN1^{nu/nu} nude mice. Briefly, 1×10⁶ tumor cells were suspended in 100 μL growth factor reduced Geltrex basement extract (9 mg/ml, Life Technologies), subcutaneously injected over the lateral aspect of the right hindlimb. Two weeks before tumor inoculation, the mice were placed on an alfalfa-free diet (Envigo 2018SX), to reduce chlorophyll autofluorescence. Ten days after tumor inoculation (710.4±572.2 mm³ for mean tumor volume at day 0) the mice received a single intravenous injection of Cy 5.5-labeled MANP (1 mg/kg eq. MithA) via lateral tail vein, and subjected to serial in vivo imaging at 1, 2, 4, 8, 24, 48, and 72 hours after Cy5.5-MANP injection. An Ami-X in vivo optical imaging system (Spectral Instruments Imaging, Inc. Tucson, Az) was used to localize the biodistribution of the Cy5.5-MANP injection. Images were acquired with epi-illumination through a 675±20 nm excitation filter at 75% power, and fluorescence emission recorded through a 790±20 nm filter. To maintain positioning during image acquisition, mice were anesthetized with 1-3% isoflurane vapor in 0.2 lpm oxygen delivered through a nosecone manifold. At the end of the study, the mice were euthanized by CO₂ inhalation. Due to tumor burden exceeding 1500 mm³, one mouse was euthanized at each 4 and 24 hours following Cy5.5-MANP injection; the remaining mice (n=8) were euthanized at 72 h. To localize the Cy5.5-MANP signal to specific organs the kidneys, liver, spleen, lung, ipsilateral quadriceps, brain and heart and tumor mass were dissected and positioned for ex vivo imaging. Following ex vivo imaging tumor, kidney and liver tissue was snap frozen in OCT media to visualize the vascular/extravascular distribution of Cy5.5-MANP in cryosections.

Ex vivo localization of Cy5.5 MANP was evaluated at 4, 24 or 72 hours after injection, and quantified at 72 h (n=8). The background corrected fluorescence intensities were analyzed as photon intensity/sec/cm²/sr by Aura software (v. 1.6.0) using operator-defined regions of interest (ROI) measurements. For ex vivo images, rectangular ROI were placed around the respective organs for each animal. For in vivo serial images, a ‘tumor signal’ ROI were manually drawn around the contour the subcutaneous tumor mass visible in the brightfield image; a second ‘blood-volume signal’ ROI were placed over the dorsal aspect of the head/neck region, just caudal to the ears. To isolate the extravascular accumulation of the Cy5.5-MANP, from circulating Cy5.5-MANP, the blood-volume signal was subtracted from the tumor signal for each mouse, at each time point.

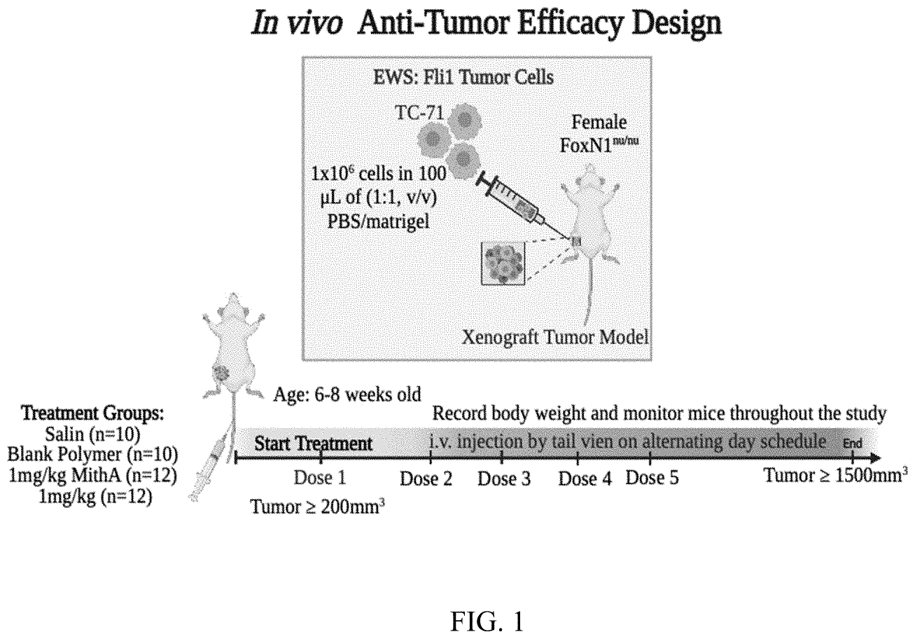

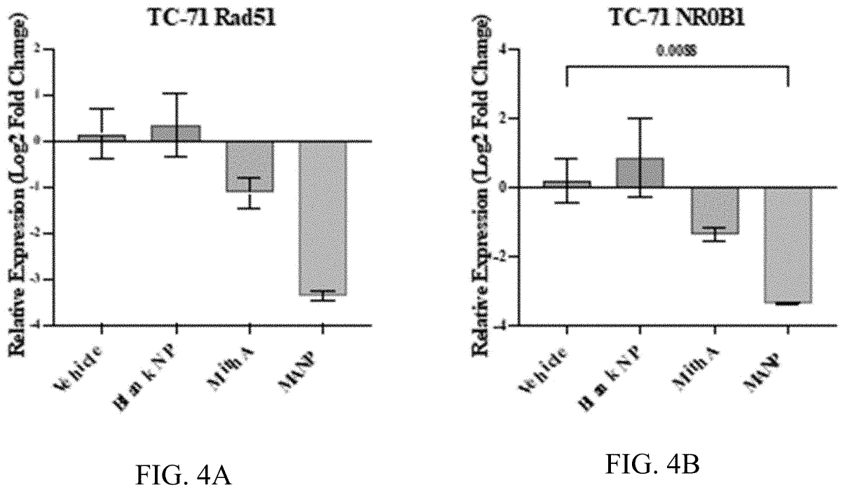

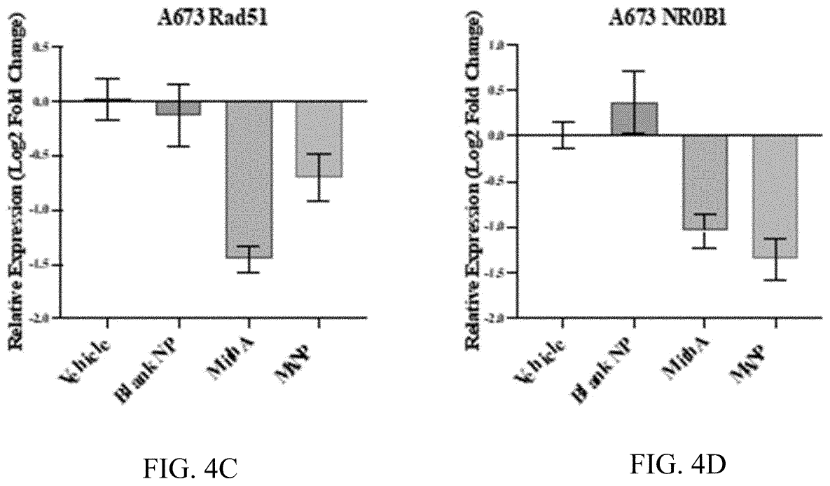

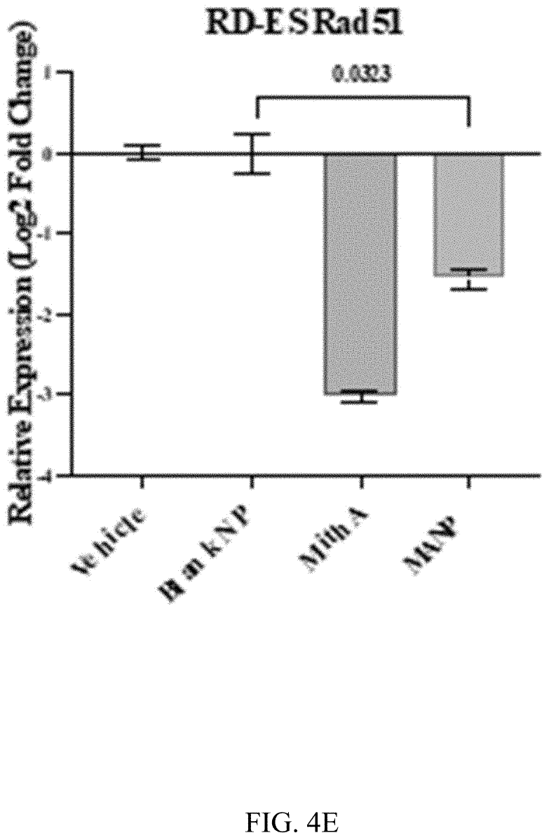

In vivo anti-tumor efficacy. Xenograft tumors of TC-71 cells were established as described above; A total of 45 mice were inoculated and engraftment rate was 97.8% (44/45) for this experiment. On alternating days following inoculation, body weight was recorded, and tumor volume was measured using digital calipers. As each mouse achieved a measurable tumor volume of 200 mm³, they were randomly allocated saline control vehicle, blank polymer (nano-formulation without MithA conjugation), 1 mg/kg MithA, or 1 mg/kg MANP (1 mg/kg eq. MithA) treatment groups until n=10/group was reached. Mice received 5 intravenous injections of the designated treatment via tail vein, given on alternating day schedule (e.g. M-W-F-Su-Tu). After treatment, tumors were allowed to grow to 1.5 cm³, at which point the mice were euthanized by CO2 inhalation (FIG. 4.1.). The tumor volume was calculated by the equation: Tumor volume=(L×W²)/2, in which L and W are the longest and shortest in tumor diameters (mm), respectively. Survival duration was calculated as the number of days for tumor volume to increase from 200 to 1500 mm³. Retrospective analysis indicated that groups of 10 animals were sufficient to detect an extension of median animal survival duration of greater than 10 days as statistically significant at α=0.05/β=0.2. FIG. 1 depicts a schematic illustration of in vivo anti-tumor efficacy study.

In vivo toxicity of MithA and MANP. Cohorts of n=6-7 C57BL/6J females, 8-10 wko were randomized to receive either intravenous or intraperitoneal administration of saline control vehicle, blank polymer, MithA, or MANP as described in TABLES 2A and 2B. Body weight loss exceeding 20%, death, morbidity, changes in behavior, and any other sign of local or systemic toxicity were recorded.

For hematology, blood was collected 24 h after the last dose by venipuncture of the tail or mandibular veins. Blood (>100 uL) was transferred to Sarstedt EDTA tubes and held on ice until differential complete blood count was performed using Abaxis VetScan HM2 hematology analyzer (Union City, Calif.). Samples were analyzed within 8 hours of collection. Giemsa-stained blood smears were also obtained for manual differentiation of granulocytes, as the instrument is not able to discriminate neutrophils, eosinophils, or basophils due to inefficient lysis of mouse granulocytes by this system.

For serum chemistry blood was collected through a 20G needle 24 h after the last dose, by cardiac puncture under isoflurane anesthesia. The blood was distributed to EDTA-coated (100 uL) or clot-activator (>200 uL) Multivette-600 tubes (Sarstedt, Newton, N.C.) respectively for hematology and serum chemistry analyses. For serum isolation, blood allowed to clot at room temperature for at least 20 minutes, centrifuged 10 kG for 10 minutes, aliquoted and frozen at −80° C. until analysis on a VetScan VS2 instrument (Zoetis, Parsippany-Troy Hills, N.J.) with Zoetis Preventive Profile Plus panel for chemistry.

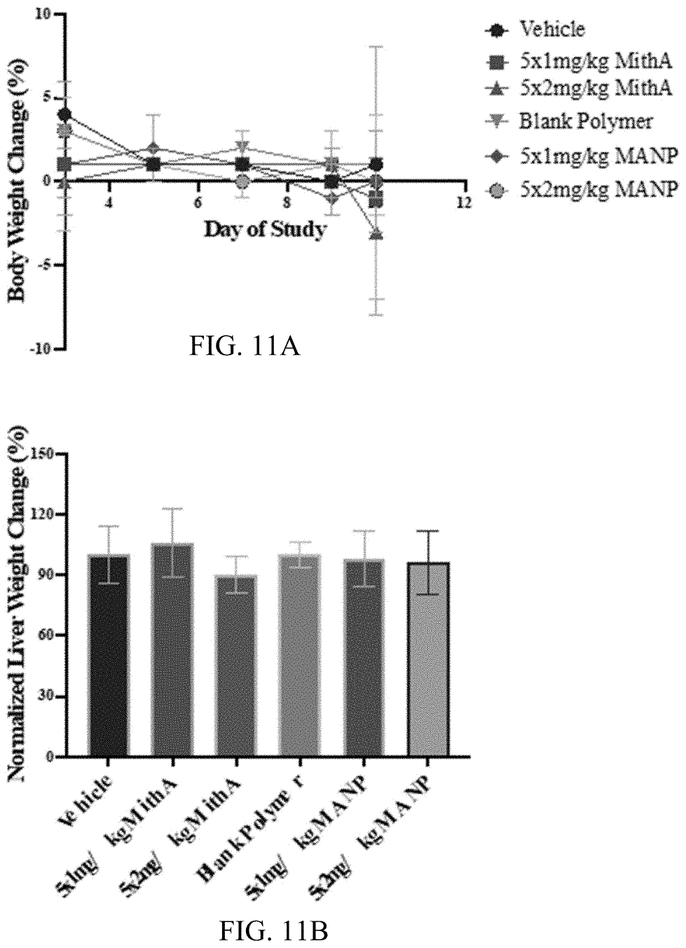

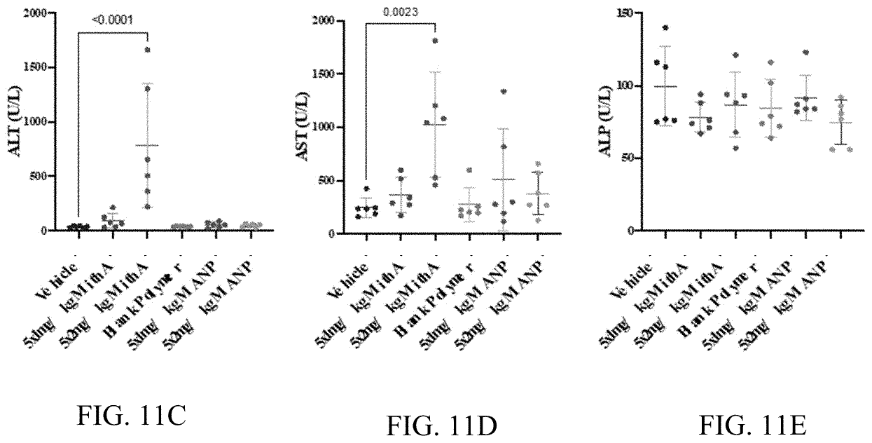

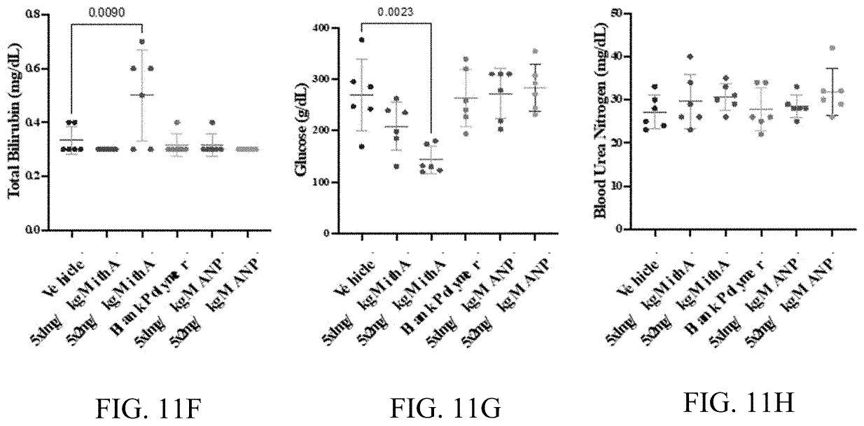

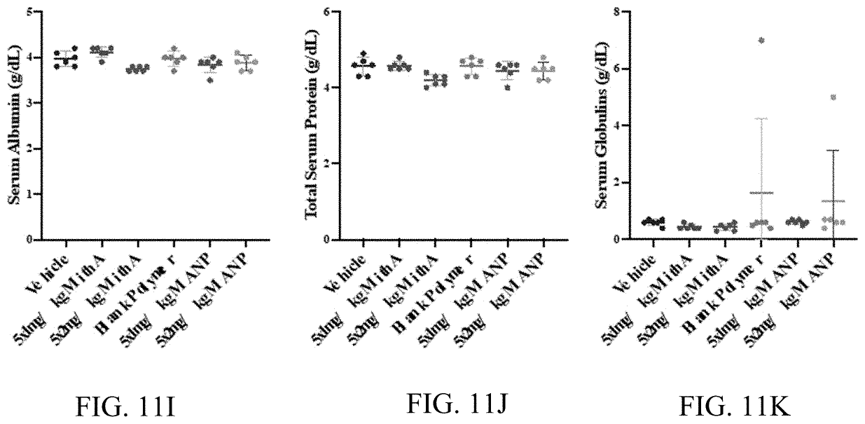

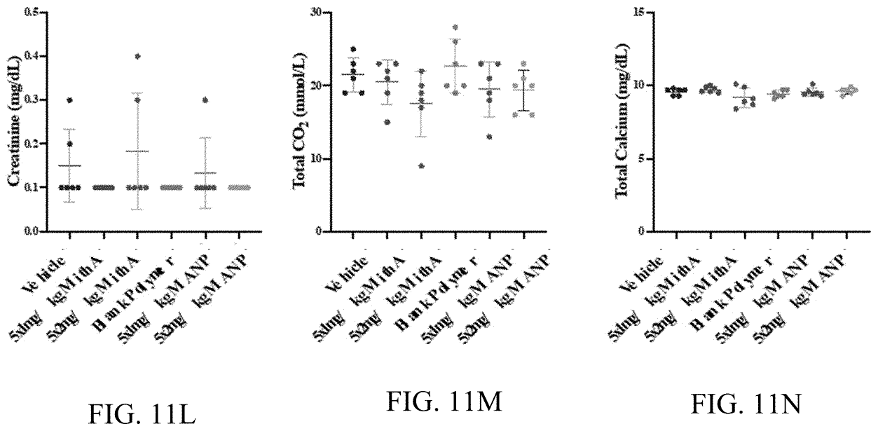

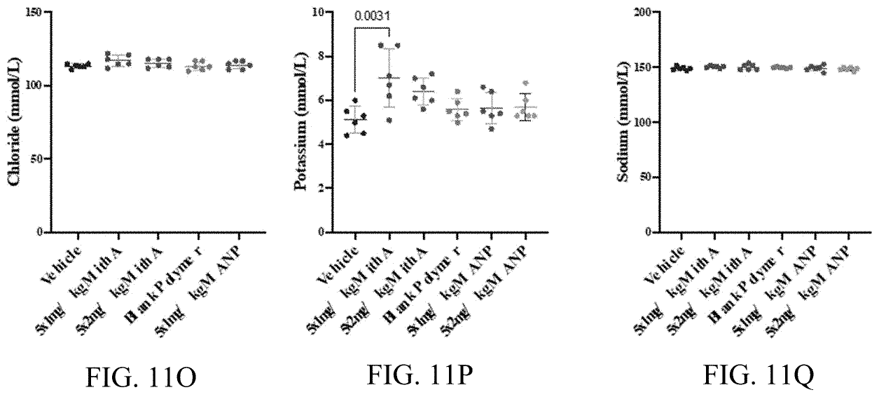

The liver was harvested for histopathological assessment, weighed, and fixed in 4% paraformaldehyde for 24 hours in the 4° C. and processed for and embedded in paraffin. After embedding, sections were cut at 5 mm thickness and mounted on poly-L-lysine-coated glass slides. Sections were stained with Ehrlich's Hematoxylin and Eosin-Phloxine solutions, Periodic Acid-Schiff (+/− pretreatment with 1% Diastase for 30 min), or Hall's Bile stain with Picrosirius red counter stain. Slides were then dehydrated, cover slipped and imaged on Nikon E800 microscope for qualitative assessment of liver injury. In vivo toxicity of Mitha and MANP study design. (A) study design for supplemental figures. (B) study design for FIGS. 11A-11Q.

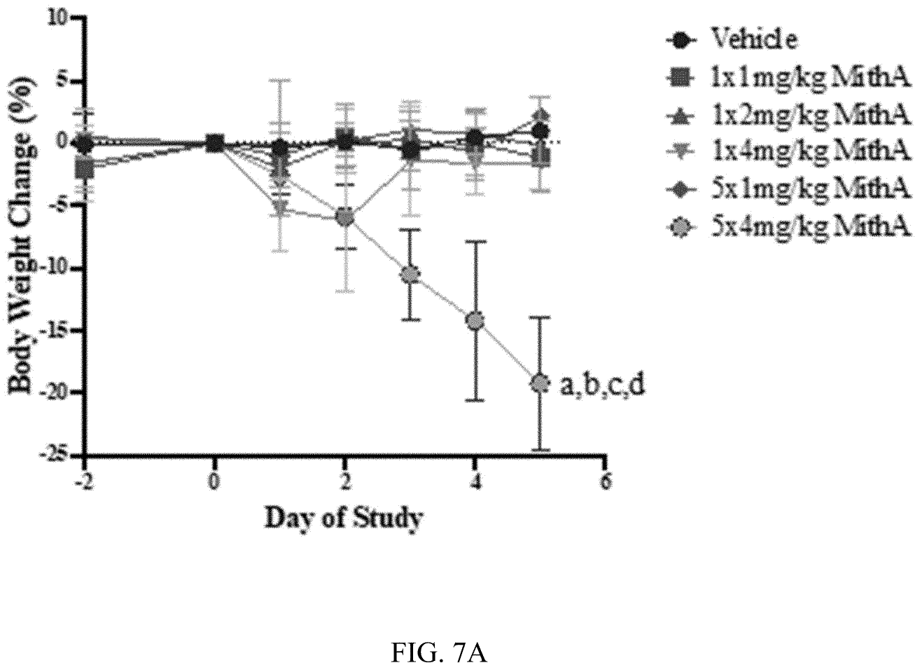

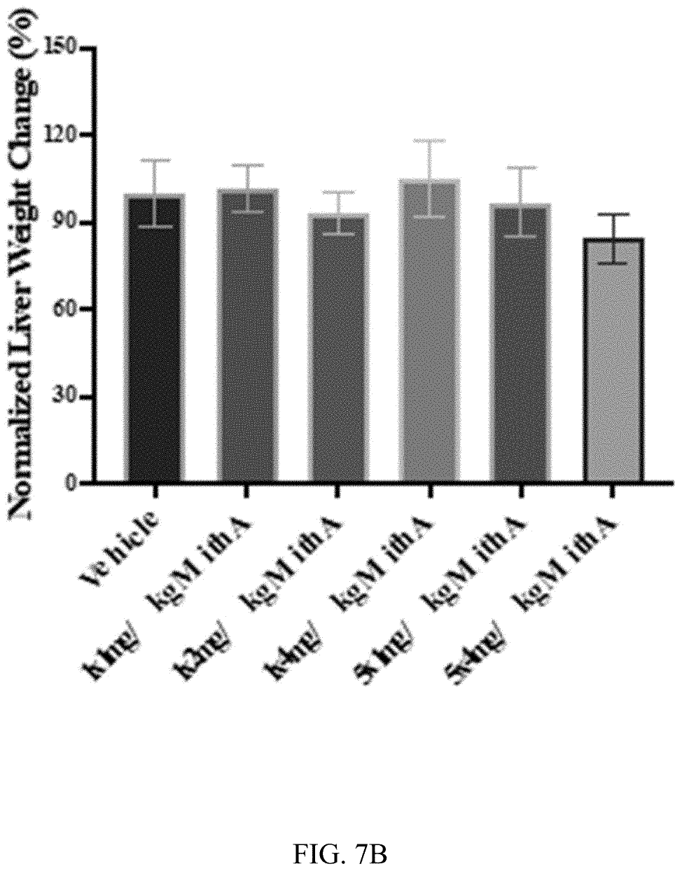

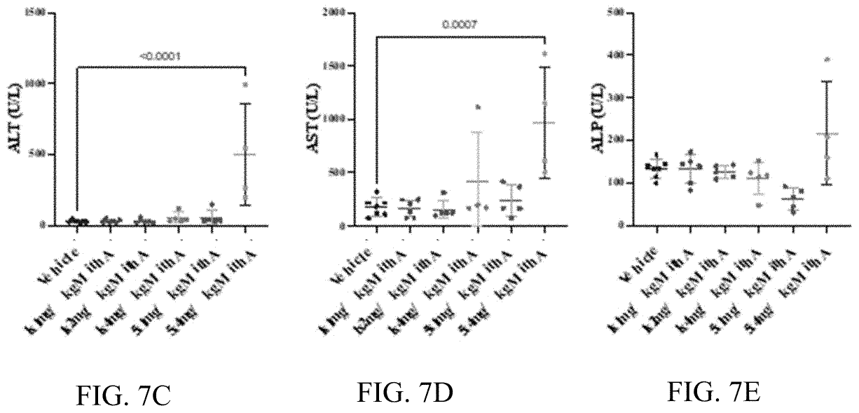

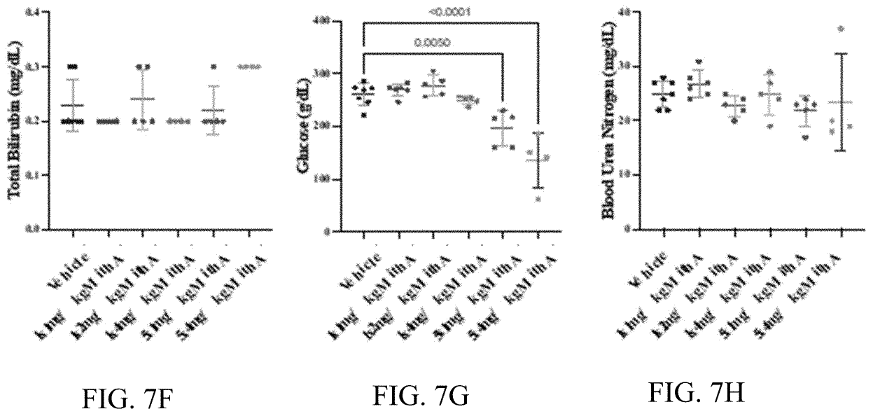

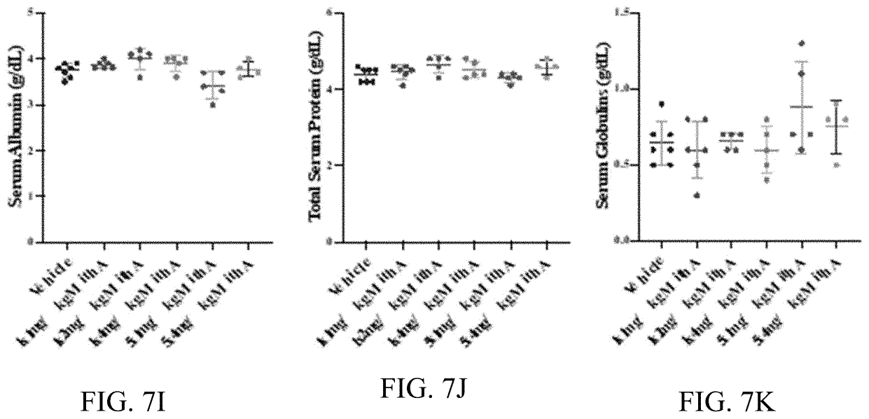

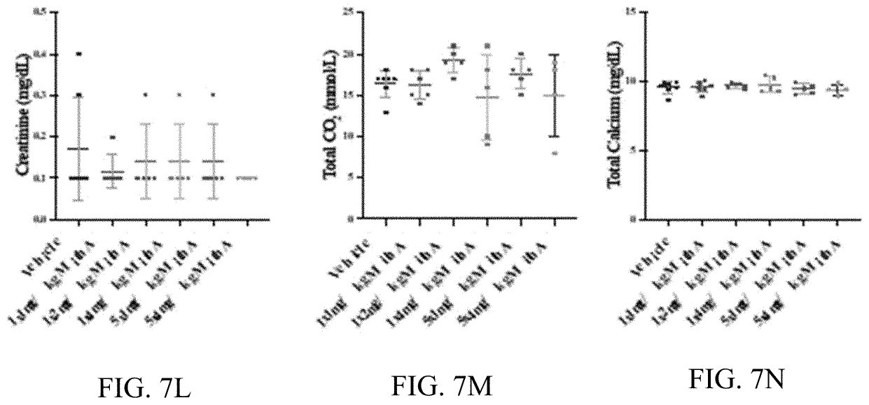

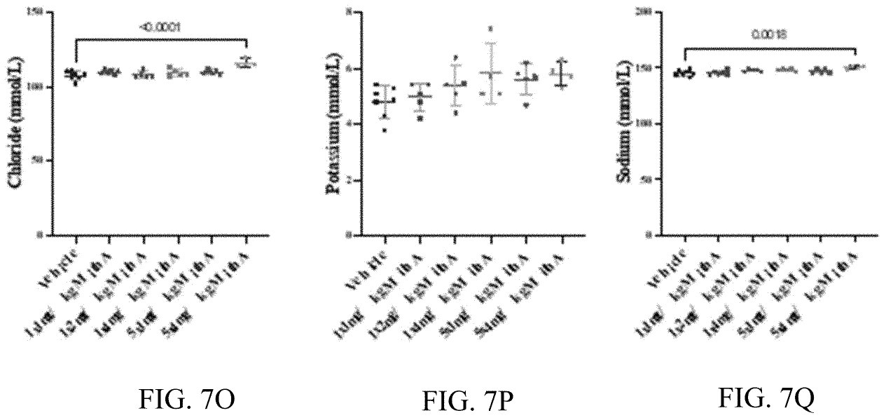

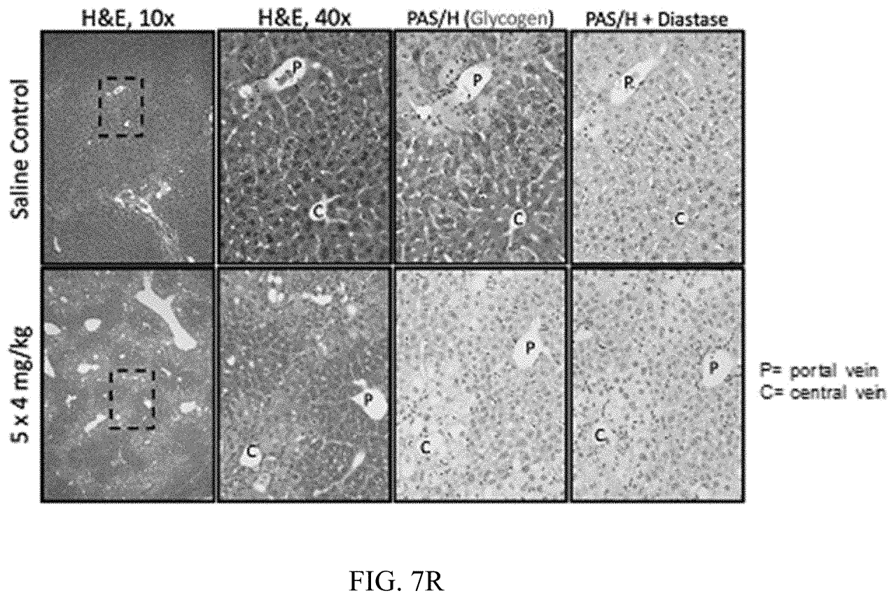

TABLES 2A and 2B In vivo toxicity of Mitha and MANP study design. Table 2A shows the study design for FIGS. 7-10. Table 2B shows study design for FIGS. 11A-11Q.

A

| Route of | | | | | | | | |
|---|---|---|---|---|---|---|---|---|
| Administration | i.p | i.p | i.p | i.p | i.p | i.p | i.v | i.v |
| Treatment | 1X | 1X1 | 1X2 | 1X4 | 5X1 | 5X4 | 1X4 | 1X4 |
| n = | Saline | mg/kg MithA | mg/kg MithA | mg/kg MithA | mg/kg MithA | mg/kg MithA | mg/kg MithA | mg/kg MANP |
| | 9 | 7 | 7 | 8 | 6 | 6 | 10 | 10 |
| Frequency | Once | Once | Once | Once | Everyday | Everyday | Once | Once |

B

| Route of | | | | | | |
|---|---|---|---|---|---|---|
| Administration | i.v | i.v | i.v | i.v | i.v | i.v |
| Treatment | 5X | 5X | 5X1 | 5X2 | 5X1 | 5X2 |
| n = | Saline | Blank Polymer | mg/kg MithA | mg/kg MithA | mg/kg MANP | mg/kg MANP |
| | 7 | 7 | 9 | 7 | 7 | 7 |
| Frequency | Alternating Days | Alternating Days | Alternating Days | Alternating Days | Alternating Days | Alternating Days |

SUPPLEMENTAL TABLE S4.3
Antibodies

| Primary | | | | |
|---|---|---|---|---|
| Target | Species, class/isotype | Supplier (cat #) | Application | dilution |
| β-actin | Mouse, IgG1 | Sigma (A1978) | WB | 1:2000 |
| Rad51 | Rabbit, Recombinant | Abcam (ab133534) | WB | 1:1000 |
| NR0B1 | Rabbit, Recombinant | Sigma (AV45592) | WB | 1:1000 |
| Secondary Antibodies | | | | |
| Target | Host Species, Conjugate | Supplier (cat #) | Application | dilution |
| Mouse IgG | Donkey, IRDye 800CW | LI-COR (926-32212) | WB | 1:15000 |
| Rabbit IgG | Donkey, IRDye 680RD | LI-COR (926-68073) | WB | 1:15000 |

SUPPLEMENTAL TABLE S4.4
RT-qPCR Primers

| | | NCBI- | | For. | | For. |
|---|---|---|---|---|---|---|
| Gene | Gene | [symbol] | Forward | length | For. | Tm |
| (mRNA) | symbol | [symbol] | Primer (5′-3′) | (bp) | % GC | [symbol] |
| [symbol] | [symbol] | NM_ [symbol] | AGCACAAATCAAGCGCAGG | 19 | [symbol] | [symbol] |
| [symbol] | [symbol] | NM_ [symbol] | CAACCCATTTCACGGTTAGAGC | 22 | 50 | 66.0 |
| [symbol] | [symbol] | NM_ [symbol] | CATGTACGTTGCTATCCAGGC | 21 | [symbol] | 63.8 |
| | Rev. | | Rev. | | mRNA | |
| Reverse | length | Rev. | Tm | Amplicon | [symbol] | |
| Primer (5′-3′) | (bp) | % GC | [symbol] | Size (BP) | (5′-3′) | |
| GAAGCGCAGCGTCTTCAAC | 19 | 57.9 | 60.0 | 150 | [symbol] | |
| TTCTTTGGCGCATAGGCAACA | 21 | 47.6 | 62.0 | 107 | [symbol] | |
| CTCCTTAATGTCACGCACGAT | 21 | 48.0 | 62.2 | 250 | [symbol] | |

[symbol]

indicates data missing or illegible when filed

Western Blotting. Expression Western blotting was performed to assess the expression of NR0B1 (EWS:Fli1 target gene) and Rad 51 (DNA homologous recombination repair HRR) at protein level as provisory described in Lin et al 2021. Briefly, cells were grown to ˜70% confluence and exposed to 10 nM of MithA/MANP (final concentration) or DMSO, (0.01% v/v) vehicle and blank polymer for 24 hours. Lysates were collected in cold RIPA lysis buffer supplemented with 1×HALT protease/phosphatase inhibitor cocktail (Thermo Scientific) and clarified by centrifugation. Protein concentration of the supernatant was determined by BCA assay (Thermo Scientific), and then diluted in 4× loading buffer (Licor Inc., Lincoln, Nebr.) containing 1× NuPAGE reducing agent (Life Technologies). Prior to electrophoresis, samples were denatured by incubating at 70° C. for 10 minutes. Samples were then resolved (25 μg/lane) on 4-20% gradient SDS-PAGE gels and transferred to low-fluorescence PVDF membranes (Bio-Rad Inc., Hercules Calif.). Transfer efficiency and equivalence of total protein load was assessed using Revert 700 Total Protein Stain kits (Licor Inc). Membranes were then washed, blocked in Odyssey Blocking Buffer (PBS) (Licor Inc), and incubated with primary antibodies (Table S4.3 above) overnight at 4° C. diluted in blocking buffer. Subsequently, membranes were washed and incubated for 90 minutes at room temperature using species-appropriate fluorophore-conjugated secondary antibodies and visualized on a Licor Odyssey instrument. Beta-actin was used as internal control and for normalization. Semi-quantitative densitometry was performed using ImageStudio software (Licor Inc).

Assessment of mRNA expression by RT-qPCR. Total RNA was extracted from EWS:Fli1⁺ cell cultures (TC-71, A673, and RD-ES) exposed to 10 nM of MithA/MANP (final concentration) or DMSO, (0.01% v/v) vehicle and blank polymer for 24 hours by the RNeasy technique with RNeasy columns with on-column DNAse I digestion (Qiagen, Valencia, Calif.) according to the manufacturer's instructions. The concentration and purity of the extracts were determined by UV absorbance at 230/260/280/320 nm wavelengths using a Tecan Infinite M200 spectrophotometer. Template cDNA was synthesized from 1 pg/sample of RNA extract by reverse-transcription using the Quantitect RT kit (Qiagen). Oligonucleotide primer sequences and more detailed information described in Supplemental Table S4). RT-PCR was performed using Quantitect SYBR-green qPCR reagents in a Mastercycler ep Realplex2 instrument (Eppendorf AG, Hamburg, Germany). PCR reactions were run in duplicate, and each experiment was independently replicated three times. Relative quantification of mRNA expression between MithA/MANP or vehicle/blank polymer treated cells were semi-quantitatively calculated by comparing the average threshold cycle (Ct) for the treated samples to the average Ct for untreated samples (Vehicle or blank polymer. Amplification efficiency was determined for each primer set, and the Pfaffl equation to determine relative fold change in gene expression. Beta-actin was used as the housekeeping gene for normalization.

Statistical analysis. Microsoft Excel was used to organize data and obtain descriptive statistics (mean and standard deviation). GraphPad Prism 9 software (GraphPad Software, Inc., La Jolla, Calif.) was used to calculate the IC₅₀ of MithA and MANP. For Western blot, tumor-bearing xenograft, and toxicity studies, one-way ANOVA with Bonferroni correction and multiple comparisons were performed using Prism 9. All in vitro data presented are derived from a minimum of three independent replicate experiments. For xenograft studies, survival data were used to construct Kaplan-Meier plots and statistical significance of differences between treatment groups (n=10-11/group) was determined by the log-rank test. Differences were accepted as statistically significant when the p-value was ≤0.05.

### Results

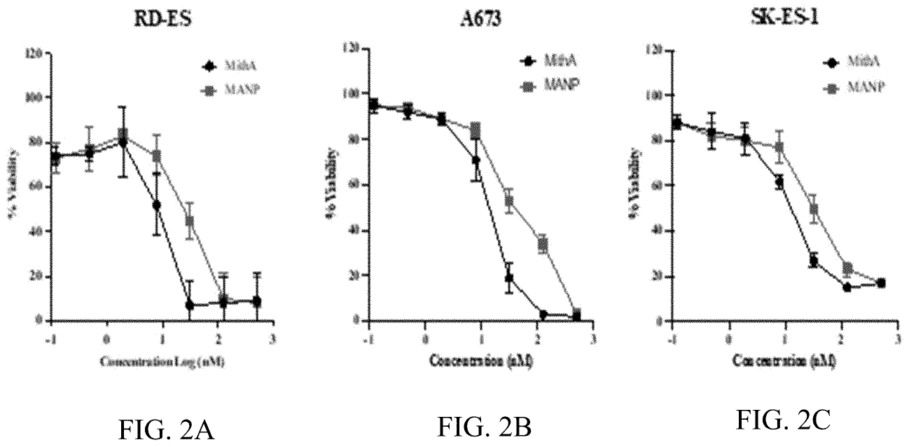

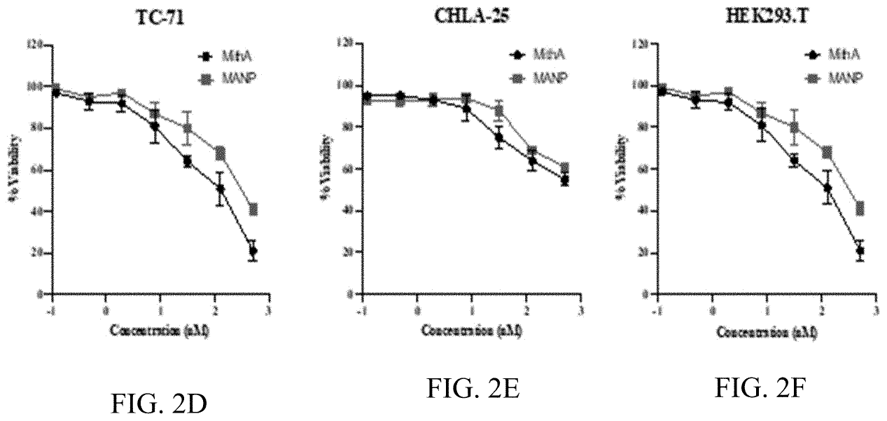

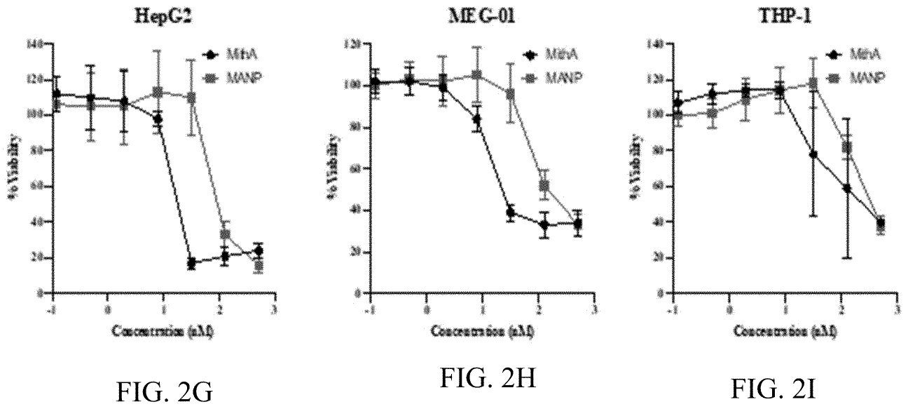

MANP potently and selectively inhibited proliferation of EWS:Fli1+ tumor cells in vitro. Potency of MithA and MANP was evaluated by in vitro viability assays, exhibited profoundly high anti-proliferative activity against EWS:Fli1+ cells, with an average IC50 of 10 nM for free MithA, and 30.8 nM for MANP (See Table 4.1 below). However, IC50 values in EWS:Erg+ cells were 348.8 nM and 537.8 nM for MithA and MANP respectively. For non-tumor cells (293.T), average IC50 was 90.4 nM and 272.2 nM for MithA and MANP respectively. Hepatoblastoma cells (HepG2), showed high sensitivity to MithA and MANP, with average IC50 of 24.94 nM for MithA and 133.8 nM MANP. The average IC50 of MithA was 233.7 nM in chronic myeloid leukemia cells (MEG-01), and 453.6 nM for MANP (FIG. 2A-2I).

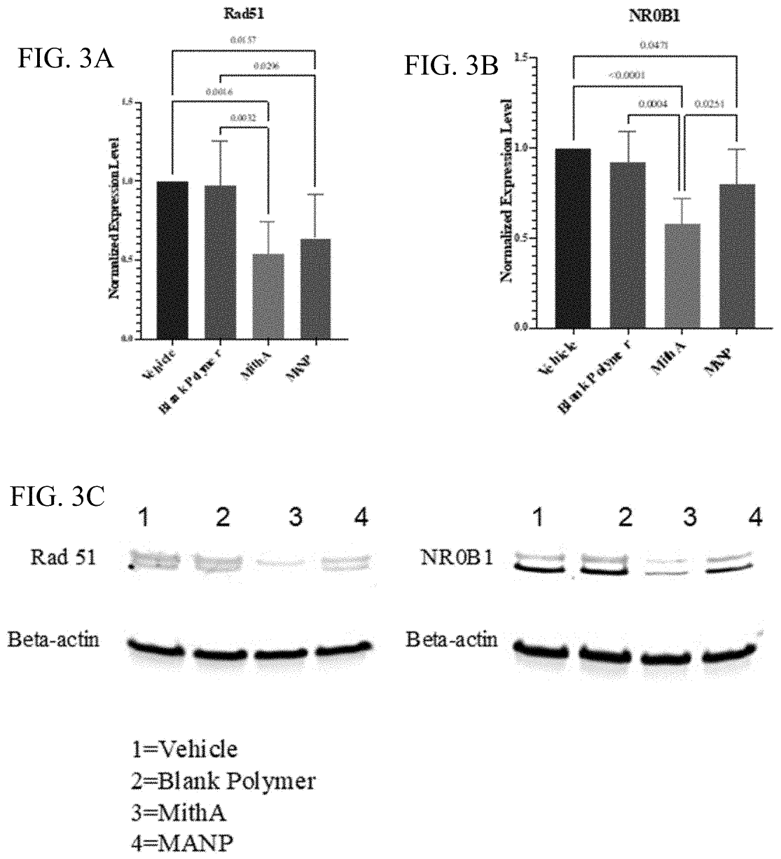

Rad 51 is an important protein in DNA DSB repair in HR pathway. It was previously shown that MithA treatment led to inhibition of DSB repair, so it was desirable to confirm this finding in MANP treated cells. NR0B1 is a downstream target of EWS:Fli1, and it physically interacts with EWS:Fli1, so this protein-protein interaction plays an important role for EWS oncogenic transformation. The potency of MithA and MANP to inhibit expression of Rad51 and NR0B1 at the protein and mRNA level were compared. Western blotting showed MithA and MANP significantly suppressed the protein expression of EWS:Fli1 at its promoter target gene NR0B1, and impaired the tumor cells DNA DSB repair capacity (HRR) with all p-value<0.0471 when compared with saline control (see e.g., FIGS. 3A-3C). However, in RT-PCR, we only detected significant inhibition of NR0B1 after MANP treatment in TC-71 cells (p=0.0088), compared with vehicle. The expression of Rad51 was inhibited in RD-ES cells (p=0.0323) compared with blank polymer (See FIGS. 4A-4E).

TABLE 4.1
Summary of Cell Lines Studies and IC₅₀ Values.

| Summary of cell lines studied and IC₅₀ values | | | Viability IC₅₀ (nM) | |
|---|---|---|---|---|
| Cell line | Tiss/Disease | Fusion | MithA | MANP |
| RD-ES | Ewing Sarcoma | EWS:Fli1 | 6.2 | 20.5 |
| SK-ES-1 | Ewing Sarcoma | EWS:Fli1 | 11.9 | 29.8 |
| A673 | Ewing Sarcoma | EWS:Fli1 | 12.4 | 41.6 |
| TC-71 | Ewing Sarcoma | EWS:Fli1 | 9.3 | 31.2 |
| CHLA-25 | PNET | EWS:ERG | 348.8 | 537.8 |
| HEK293.T | Fetal Kidney | — | 90.4 | 272.2 |
| HepG2 | Hepatoblastoma | — | 24.9 | 133.8 |
| MEG-01 | Chronic Myleoid | BRC:ABL | 37.8 | 201.9 |
| | Leukemia | | | |
| THP-1 | Acute Monocytic | MLL:AF9 | 233.7 | 453.6 |
| | Leukemia | | | |

Cell viability was tested measured following five days treatment with various concentrations of MithA and MANP (500-0.012 nM) for five days. Ewing Sarcoma cells expressing the EWS:Fli1 fusion were highly sensitive to MithA and MANP, while cells lacking this specificfusion were much less sensitive to MithA and MANP. (A-D) EWS:Fli1⁺ cells. (FIG. 2E) EWS: ERG cells. (FIG. 2F) Fetal Kidney cells. (FIG. 2G) Hepatoblastoma cells. (FIG. 2H) Chronic Myeloid cells. (FIG. 2I) Acute Monocytic Leukemia. Data shown are the mean technical replicates for three independent experiments with Mean±1 SD.

FIGS. 3A-3C depict MithA and MANP Down-regulated Rad51 and NR0B1. (FIG. 3A) Rad 51 (HR DNA repair) and (FIG. 3B) NR0B1 (EWS:Fli1 downstream target). (FIG. 3C) Representative blots for Rad51 and NR0B1. Bar graphs show normalized expression as mean±SD for 8 independently replicated experiments. Statistically significant differences (p≤0.05 by ANOVA) are indicated.

FIGS. 4A-4E depict quantification of Rad51 and NR0B1 mRNA Expression by RT-qPCR. FIG. 4A) TC-71 Rad 51. (FIG. 4B) TC-71 NR0B1. (FIG. 4C) A673 Rad51. (FIG. 4D) A673 NR0B1. (FIG. 4E) RD-ES Rad51. Statistically significant differences by ANOVA (p≤0.05).

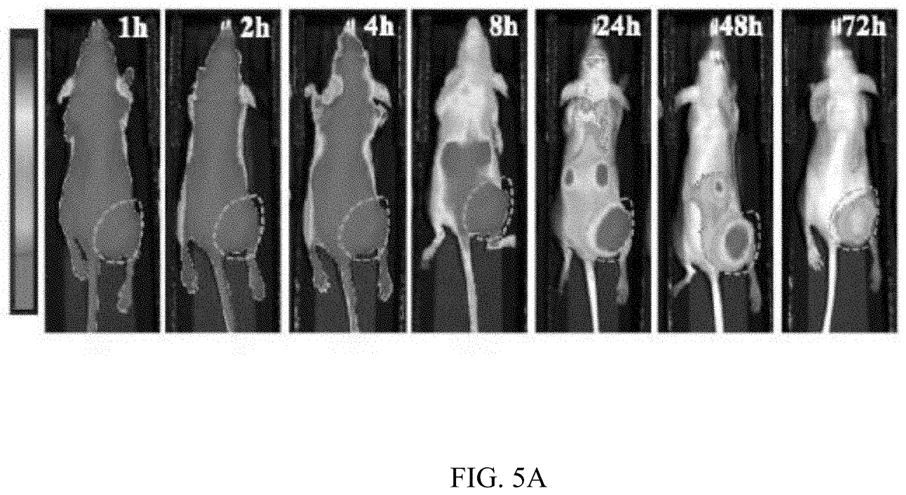

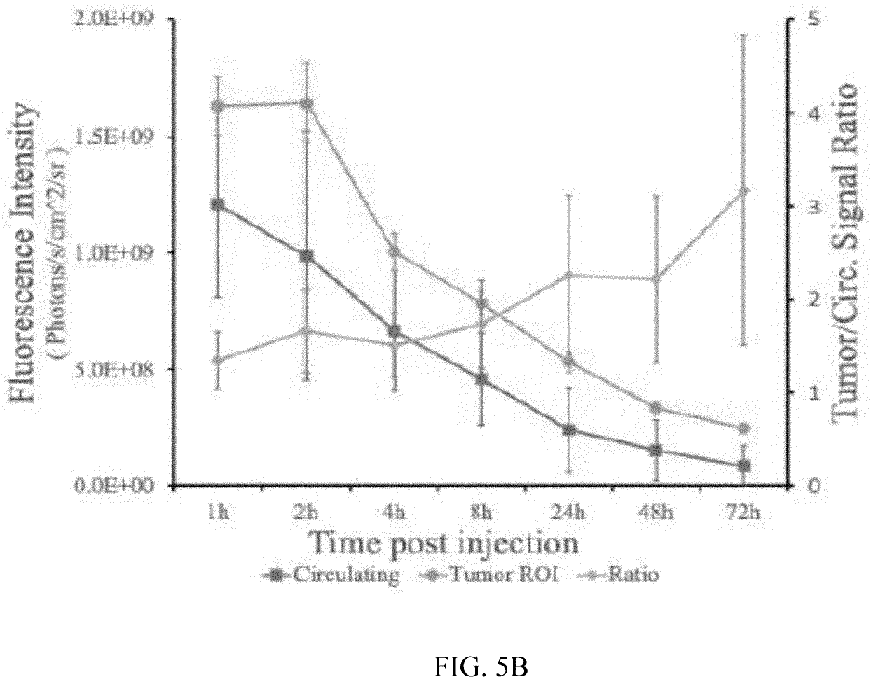

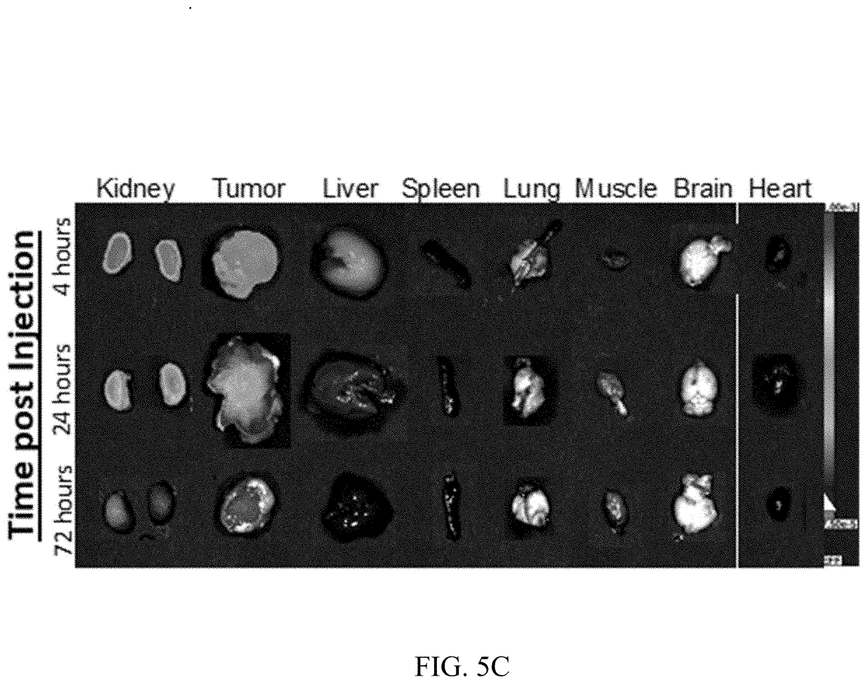

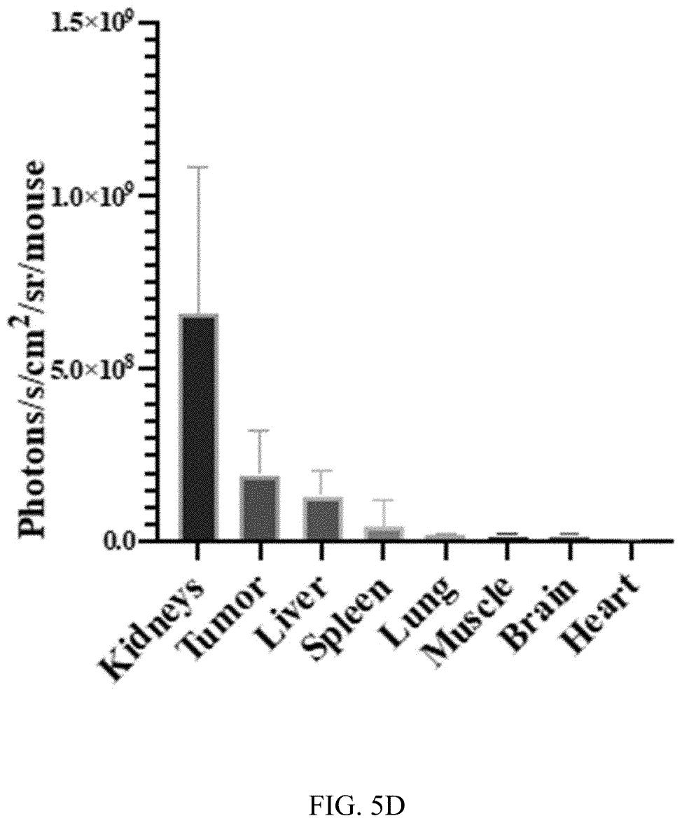

Tumor targeting and tissue biodistribution of Cy5.5-labeled MANP in TC-71 (EWS:Fli1⁺) xenograft tumor-bearing nude mice. To determine the biodistribution of MANP in tumor-bearing animals, Luo's lab conjugated MANP to a near-infrared fluorescence-emitting dye cy5.5. Following a single intravenous infusion of Cy5.5-labeled MANP showed systemic distribution of the MANP for at least 8 hours, with gradual accumulation on tumor mass over 24 to 72 hours after tail vein injection (See FIGS. 5A and 5B). Semi-quantitative analysis of the in vivo tumor uptake, Cy5.5-labeled MANP signal was gradually decreased over time in circulating and tumor (ROI) compartment, but it was retained in the tissue at a proportionally greater rate. This indicated that MANP preferentially accumulate in the tumor tissues over time after subtracting the circulating blood signals (See FIG. 5B). Ex vivo imaging of major organs at 4, 24 and 72 hours after infusion showed cy5.5-labeled MANP concentrated in the kidneys and tumor mass at these time points, gradually reducing over time (See FIGS. 5C and 5D). It is not presently clear whether this reflects extravascular retention of MANP in the kidneys or concentration and urinary clearance of the MANP or free Cy5.5 clearance through the urine. There was some signal present in the lungs at 4 hours after injection, but liver signal decreased over time, and not detectable at 72 h. Moreover, spleen, lung, muscle, brain, and heart had very minimal uptake of cy5.5-labeled MANP at any time point throughout the studies.

FIGS. 5A-5D depict Tumor Targeting and Tissue Biodistribution of Cy5.5-labeled MANP in TC-71 (EWS:Fli1⁺) Xenograft Tumor-Bearing Nude Mice.

(A) In vivo real-time imaging of TC-71 xenograft tumor-bearing nude mice treated by single i.v. injection of Cy5.5-labeled MANP (1 mg/kg eq. Free MithA). Grey dashes outlined the area with tumor (B) Semi-quantitative tumor fluorescence intensity of ROI (dashed grey) of TC-71 xenograft tumor-bearing nude mice at different hours after single i.v. injection of Cy5.5-labeled MANP with background-corrected. X-Y graph showed n=8-10 mice at each time point with Mean±1SD. (C) Representative ex vivo images from mice sacrificed at 4, 24, or 72 h after single i.v. injection of 1 mg/kg MANP, with signal intensity indexed to a common LUT. (D) Ex vivo semi-quantitative fluorescent intensity of tumors and major organs at 72 h post-injection, with background-corrected and mean±1 SD (n=8) of photons/s/cm²/sr.

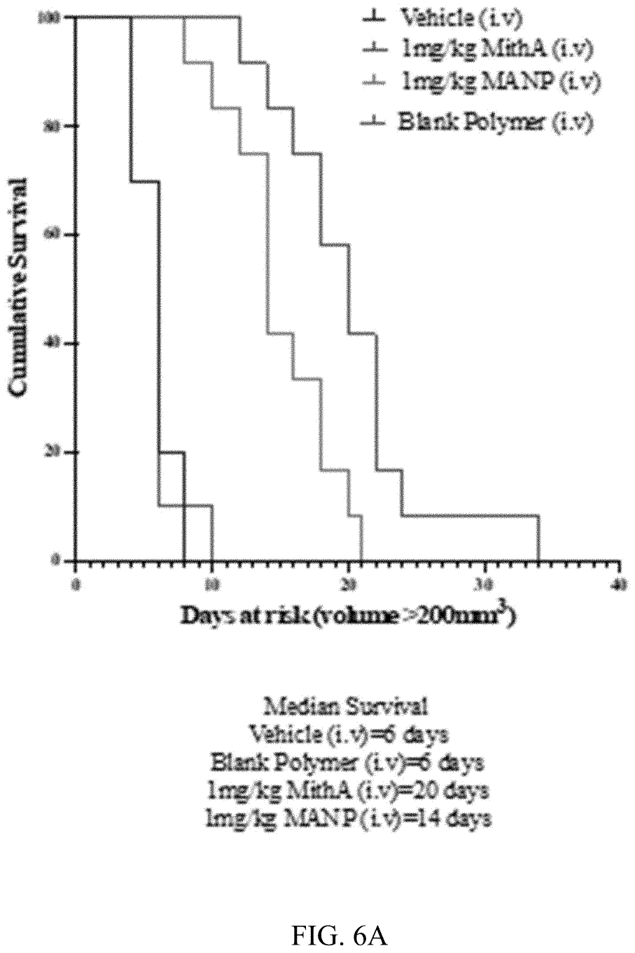

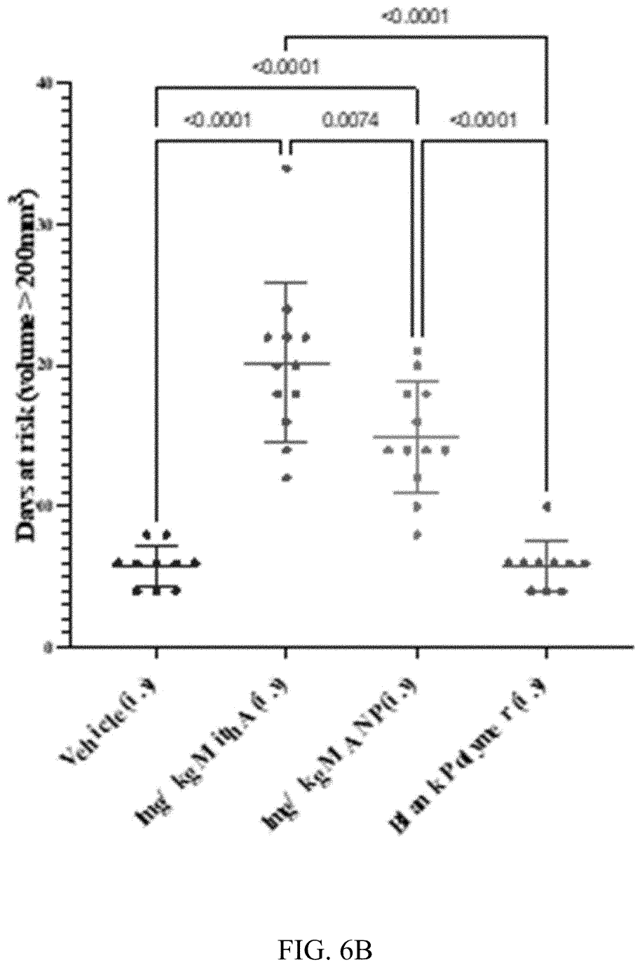

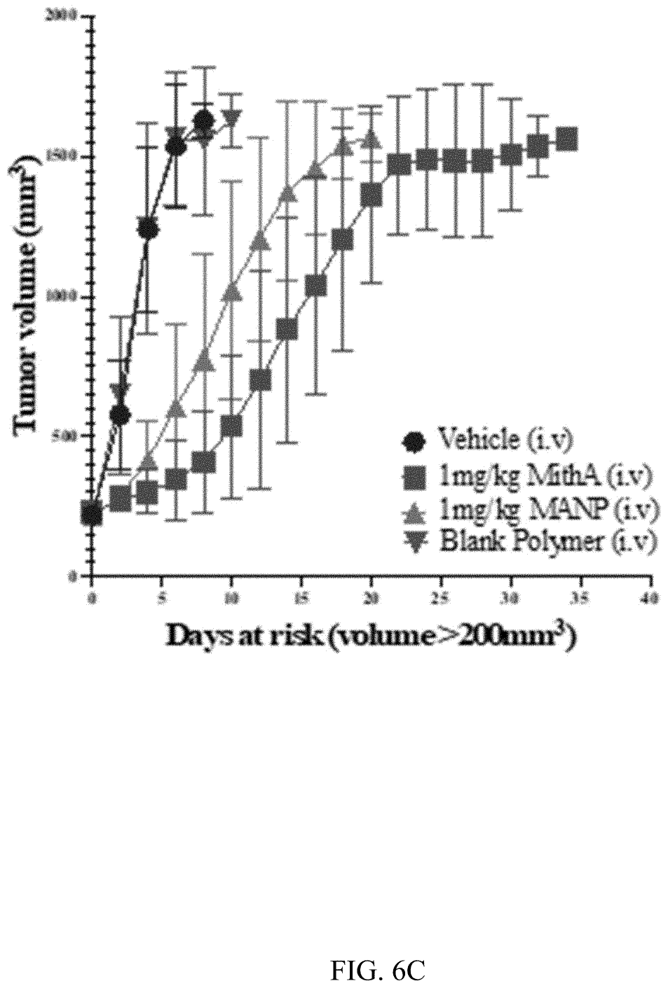

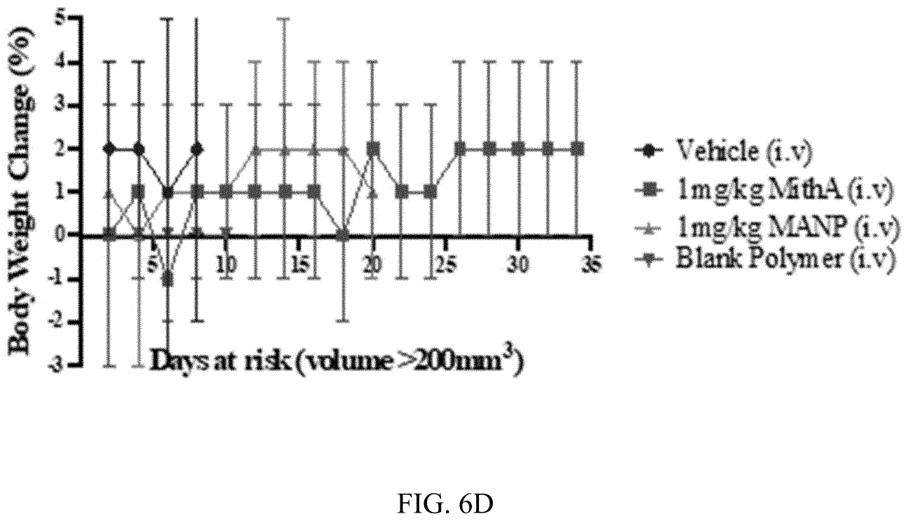

Anti-tumor efficacy of MithA and MANP in TC-71 (EWS:FIi1⁺) xenograft tumor-bearing nude mice. Treatment started at the tumor sizes of 200 mm³, five intravenous injection of 1 mg/kg MithA/MANP or saline vehicle/blank polymer on alternating days. There was no outward any signs of toxicity, or deaths under these treatments, and no loss of body weight was observed (FIG. 4.5.D). MANP suppressed tumor regrowth compared with saline vehicle and blank polymer groups (FIG. 4.5.C). Tumor bearing mice in both control groups (saline, ranged 4-8 days; blank polymer, ranged 4-10 days) had median survival of 6 days. (FIG. 4.5.B). MANP (1 mg/kg) significantly prolonged the median survival to 14 days (ranged 8-22 days, p<0.0001). However, free MithA (1 mg/kg) significantly extended the median survival to 20 days (ranged 12-35 days), significantly longer than vehicle, blank polymer or MANP treated mice (p0.0050) (FIGS. 6A and 6B).

FIGS. 6A and 6B depict Anti-tumor Efficacy of MithA and MANP in TC-71 (EWS:Fli1⁺) Xenograft Tumor-Bearing Nude Mice. Tumors were allowed to grow 200 mm³ (At risk day 0) prior to initiation of the respective treatments of 5 dose i.v. injections on alternating days. Tumors and body weight were measured until tumor volume reached 1500 mm³. (FIG. 6A) Kaplan-Meier plot of animal survival duration. (FIG. 6B) Mean survival duration. (FIG. 6C) Mean tumor volume±standard deviation. (FIG. 6D) Body weight change (%). Data are expressed as mean±1SD, n=10/group. Statistically significant differences by ANOVA.

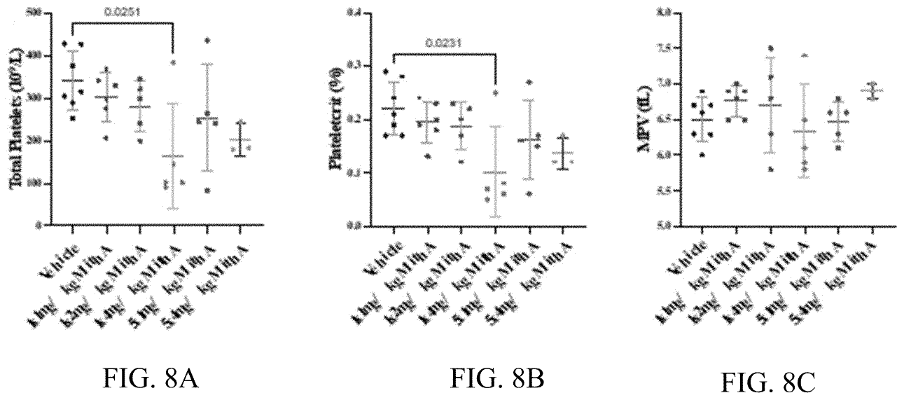

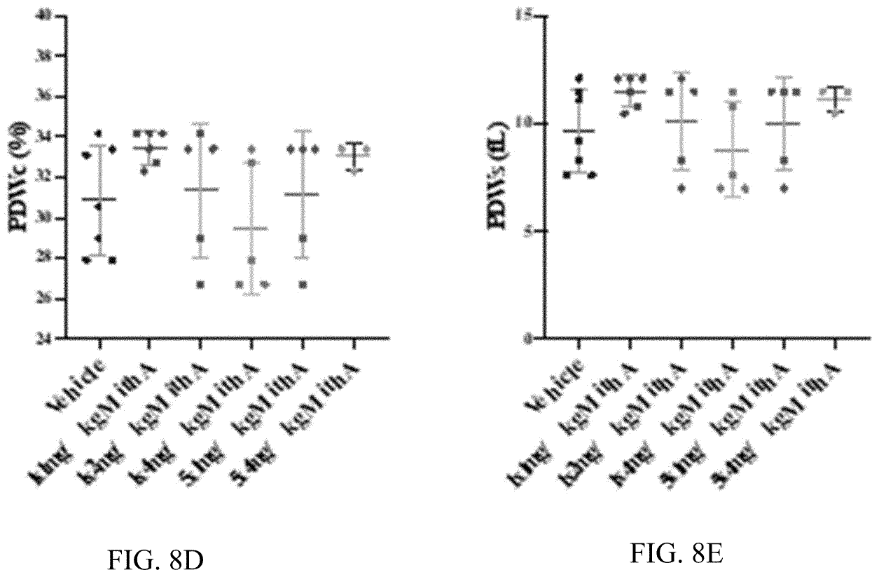

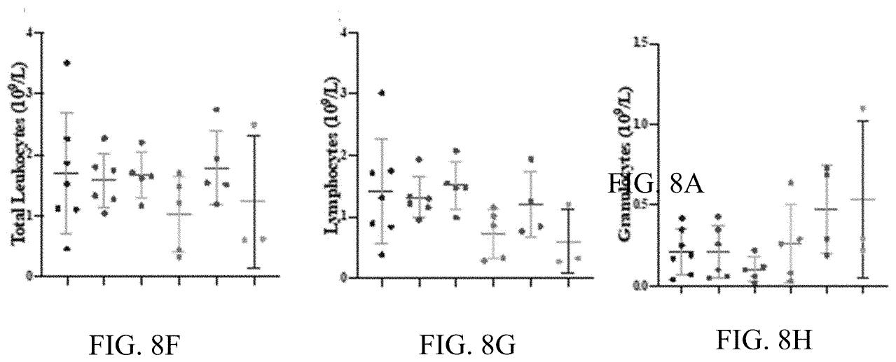

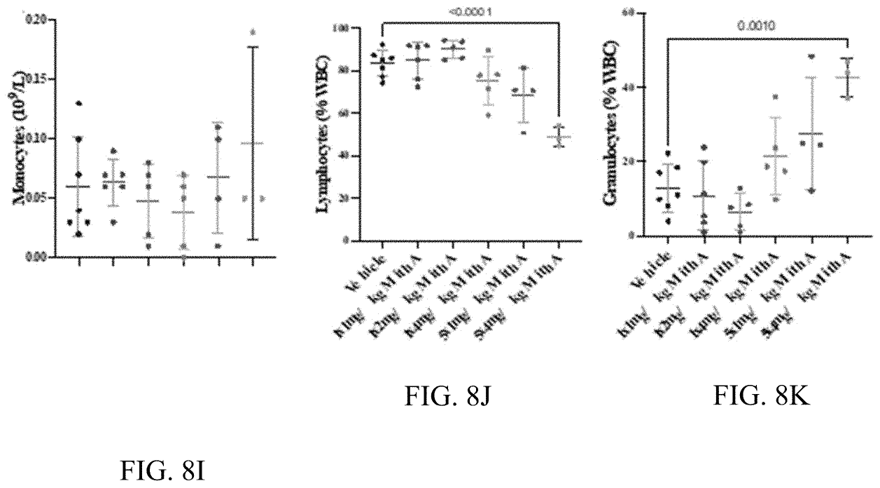

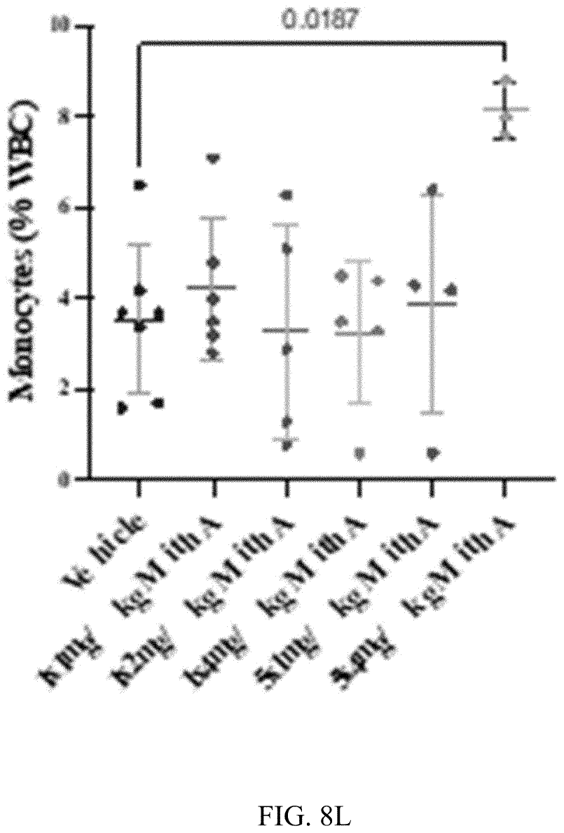

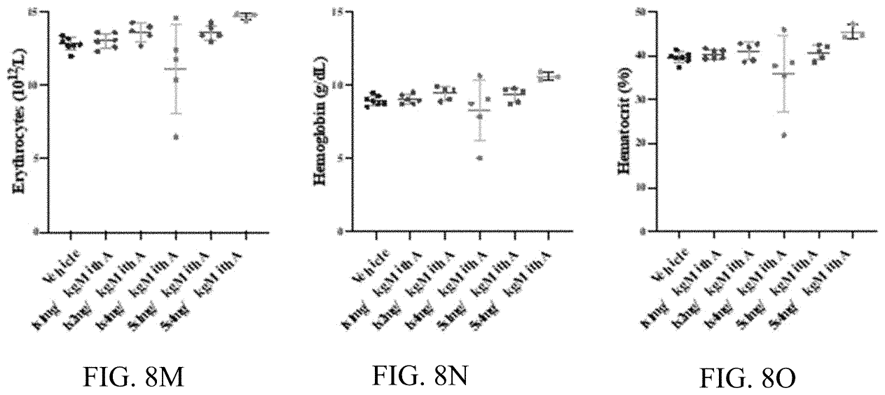

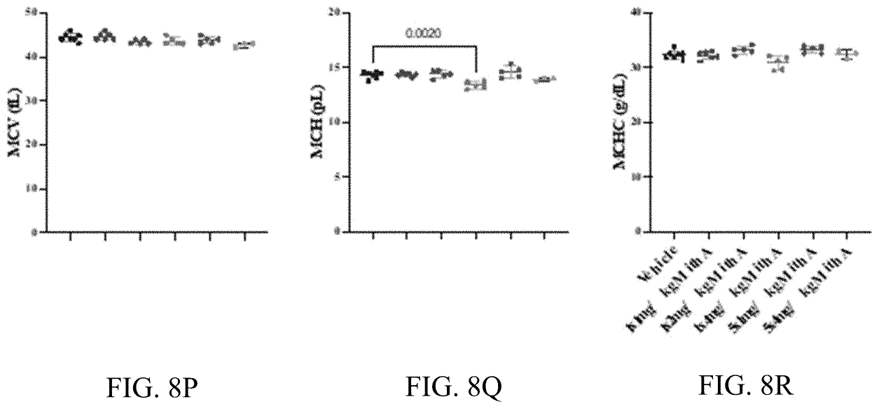

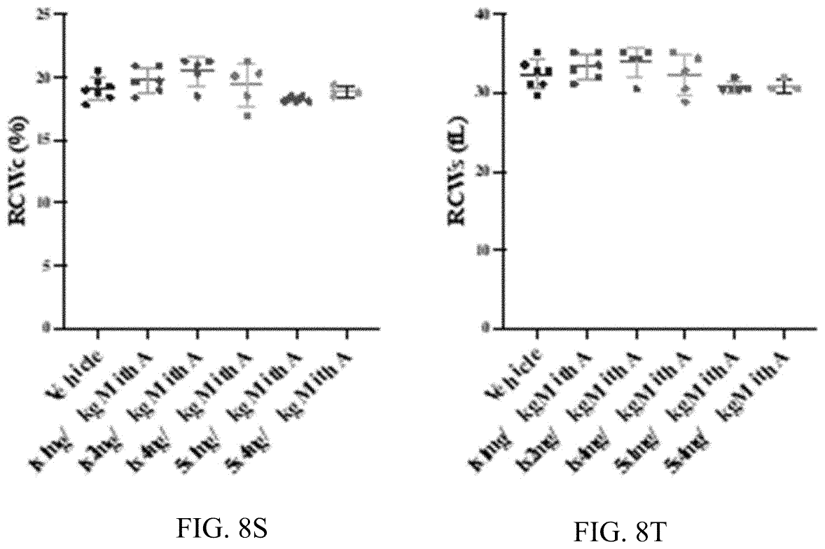

Establishing a murine model of MithA-induced toxicity in C57BL/6J mice. In this study, we first administrated MithA via intraperitoneal (i.p) injection at single dose, or 5 consecutive doses of 1 mg/kg, and 4 mg/kg of body weight. Mice tolerated a single i.p. injections of MithA at 1 mg/kg, and 4 mg/kg with no significant weight loss was observed in these groups compared with saline vehicle (FIG. 7A). Similarly, single i.p. injections of MithA did not cause any toxicity by serum chemistry (FIG. 7C-Q) or hematology assays in mice receiving 1 or 2 mg/kg doses (FIG. 7A-T). Similarly, there were no significant changes in serum in the single i.p. 4 mg/kg MithA group, but in hematology assays, significant reductions of total platelet count (p=0.0251), plateletcrit, and mean corpuscular hemoglobin (MC, p=0.002) were observed, relative to saline vehicle treated controls (See FIGS. 7A-7R and FIGS. 8A-8T)

When MithA was administered on 5 consecutive days by i.p. at 1 mg/kg dose, we observed no significant toxicity observed by serum chemistry or hematology studies, except there was a decreased in glucose level (p=0.005) compared to saline vehicle group (FIGS. 7A-7R and FIGS. 8A-8T). Interestingly, MithA toxicity became apparent when was given at a higher concentration of 4 mg/kg on 5 consecutive days via i.p injection. One mouse died in the cage before the last dose, and one mouse found to be moribund on the day of harvest (day 6 of the study). The surviving mice showed progressive and significant body weight loss (p=0.0016) and showed liver weight reduction compared to saline vehicle group (FIG. 7.A-B). Furthermore, serum collected from these mice receiving five doses of 4 mg/kg MithA showed a 16.9-fold increase of alanine transaminase (ALT, p<0.0001), and 5.4-fold increase of aspartate aminotransferase (AST, p=0.0007), and 1.9-fold decrease in glucose level (p<0.0001) relative to compared to saline vehicle group. Histologically, the livers from these mice showed periportal necrosis with central fatty degeneration, glycogen depletion and bile acid retention (FIG. 7R). Hematology results showed no significant difference, relative to controls, of total white blood cell (WBC) count. However, increased granulocytes (% Gran P=0.001) and monocytes (% MON p=0.0187), and decreased lymphocytes (% LYM p<0.0001) compared to saline vehicle group. Also, there were no significant changes in red blood cell (RBC) parameters in 5×4 mg/kg MithA (i.p.) treated mice (FIGS. 8A-8T).

FIGS. 7A-7R depict MithA Liver Toxicity Profile in Female C57BL/6J Mice. Mice were randomized to receive i.p. injections of saline vehicle or MithA at corresponding treatment design (see Table S2 for more details). Blood was collected 24 h after the last dose and prepared for blood chemistry. (A) Body weight change (%). Statistically significant differences by ANOVA (p≤0.05) indicated by mean body weight change (%) of 5×4 mg/kg vs a=vehicle; b=1×1 mg/kgMithA; c=1×2 mg/kg MithA; and d=5×1 mg/kg MithA. (B) Normalized liver weight change (%) calculated by liver (g)/body weight (g)×100%. Data are expressed as mean±1SD, n=6-9/group. Statistically significant differences by ANOVA (p≤0.05). Representative data for serum levels of (7C) Alanine transaminase. (D) Aspartate aminotransferase (7E) Alkaline phosphatase. (7F) Total bilirubin. (7G) Glucose. (7H) Blood urea nitrogen. (7I) Serum albumin. (7J) Total serum protein. (7K) Serum globulins. Statistically significant differences by ANOVA (p≤0.05). Representative data for serum levels of (7L) Creatinine. (7M) Total CO₂. (7N) Total Calcium. (7O) Chloride. (P) Potassium. (7Q) Sodium. (7R) Liver histopathology (FFPE). Statistically significant differences by ANOVA (p≤0.05).

FIGS. 8A-8T depict MithA Hematology Profile in Female C57BL/6J Mice.

Mice were randomized to receive i.p. injections of saline vehicle or MithA at corresponding treatment design (see Table S2 for more details). Blood was collected 24 h after the last dose and prepared for complete blood count tests. Representative data for serum levels of (8A) Total Platelets. (8B) Plateletcrit (%). (8C) Mean platelet volume. (8D) Platelets distribution width. (8E) PDWs. Statistically significant differences by ANOVA (p≤0.05). Representative data for serum levels of (8F) Total Leukocytes. (8G) Lymphocytes. (8H) Granulocytes. 8(I) Monocytes. (8J) Lymphocytes (% WBC). ((8K) Granulocytes (% WBC). (8L) Monocytes (% WBC). Statistically significant differences by ANOVA (p≤0.05). Representative data for serum levels of (8M) Erythrocytes. 8(N) Hemoglobin. (8O) Hematocrit (%). (8P) Mean corpuscular volume. (8Q) Mean corpuscular hemoglobin. (8R) Mean corpuscular hemoglobin concentration. (8S) RCWc (%). (8T) RCWs. Statistically significant differences by ANOVA (p≤0.05).

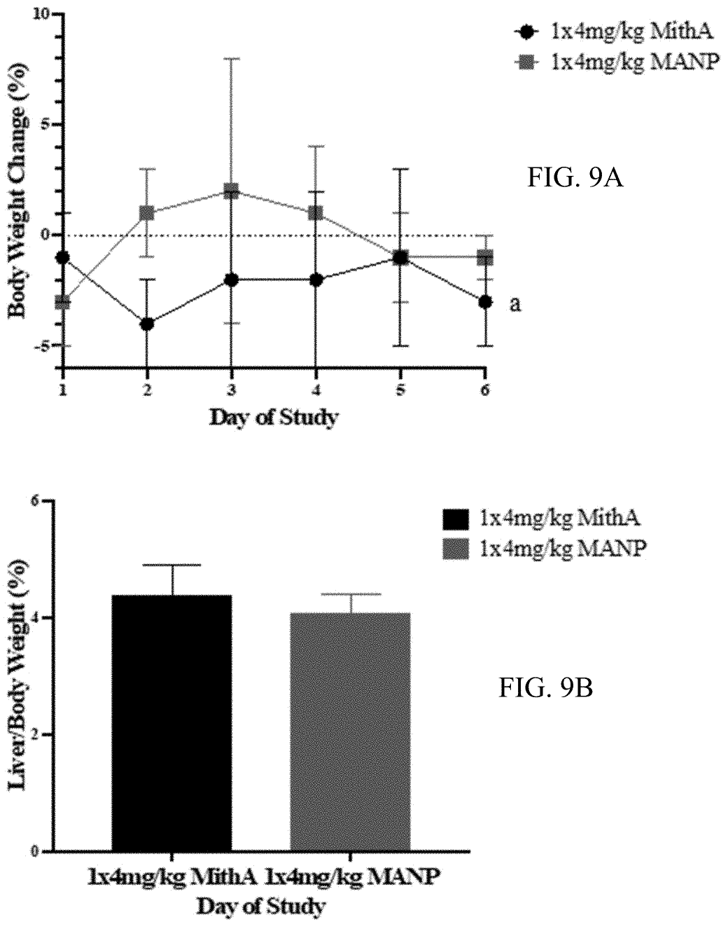

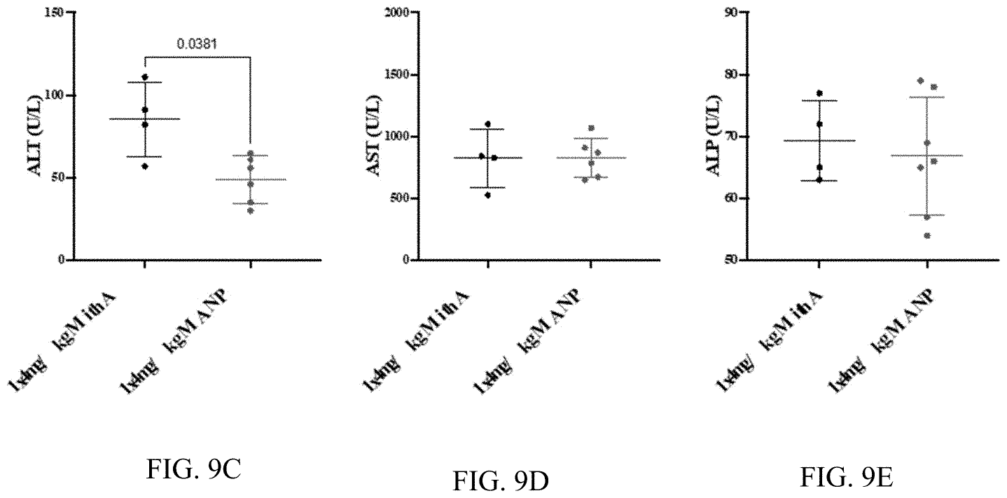

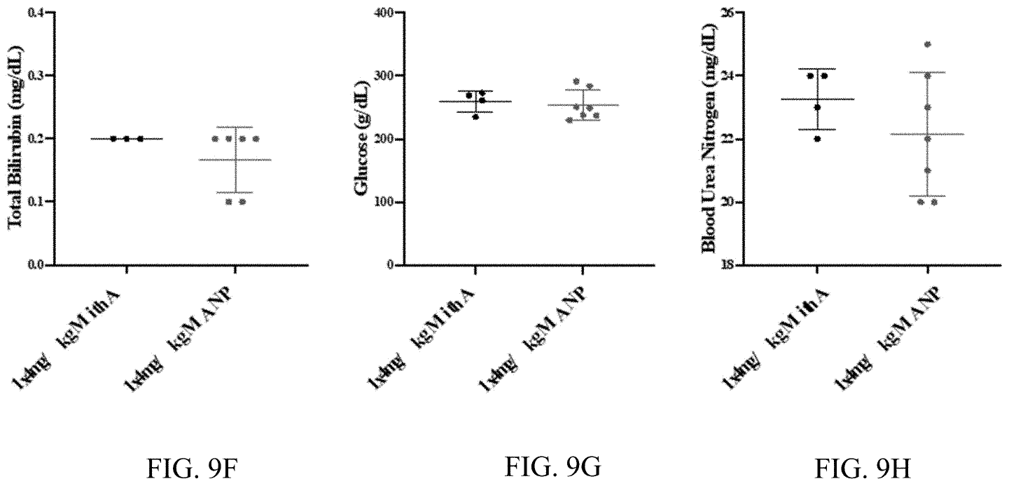

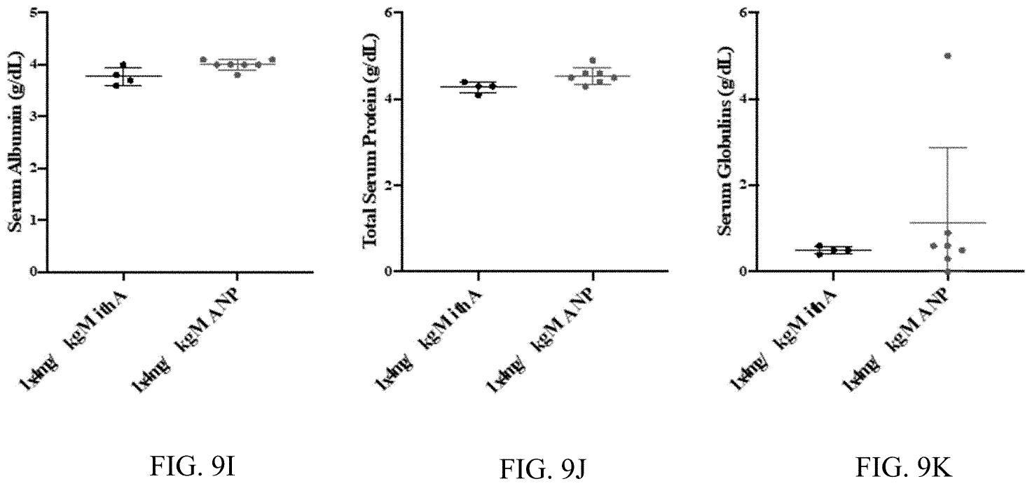

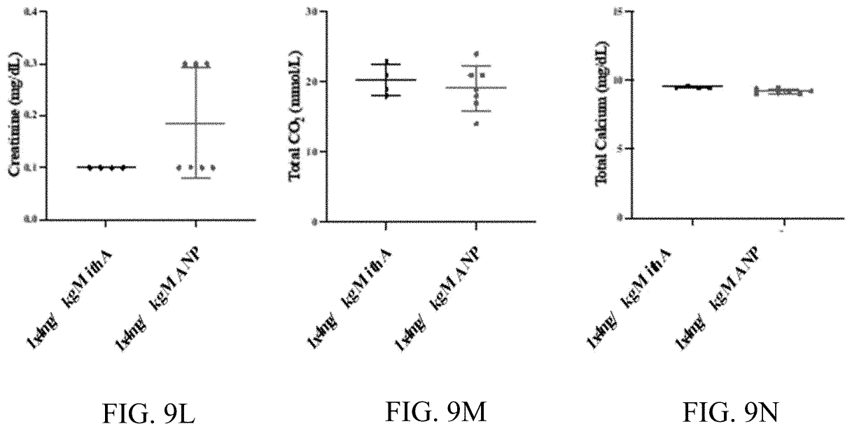

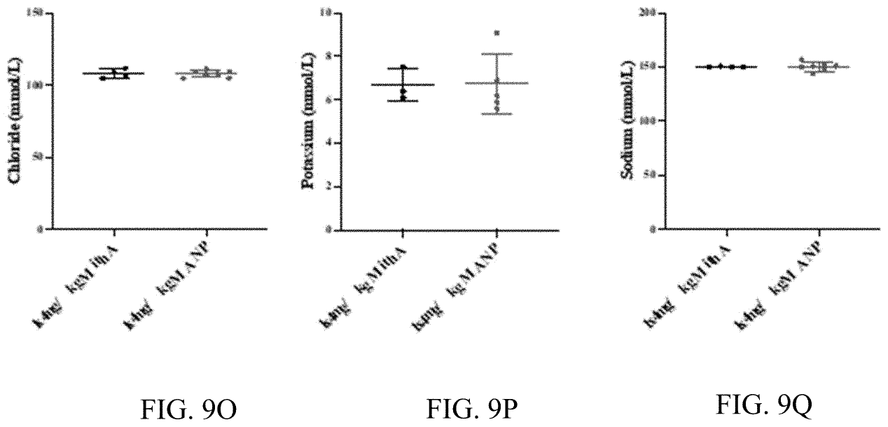

MANP alleviated MithA-induced liver toxicity and platelet dysfunction. Having established that c57BL/6j mice recapitulate MithA-induced toxicities observed in humans, we next sought to test whether our nano-formulation of MithA (MANP) could mitigate the MithA-induced toxicity. As the MANP are unlikely to passively enter the circulation following i.p. injection, we moved to an intravenous injection approach to ensure that the MANP would be efficiently delivered to the tumor. Using a similar experimental design, we sought to compare the toxicity of a series of intravenous injections of 4 mg/kg free MithA to an equivalent dose of MANP. However, this experiment was aborted upon observation of severe malaise and animal death withing 24 hours of a single i.v. injection of both free MithA and MANP. Within 48 hours of the injection, 6/8 mice became moribund and expired (6/8; 75% death rate). In contrast only 2/10 mice died in the MANP group. The remaining animals were euthanized, and serum and liver tissue were collected approximately 48 hours after the drug was administered. Unfortunately, hematology studies were not possible. We observed a significant decrease in body weight (p=0.048) (FIG. 9A), and a 1.7-fold increase in ALT liver enzyme level (p=0.0381) in comparison to the same equivalent dose of MANP treated group (FIG. 9C). The acute toxicity after drug administration made the sample size much smaller at the endpoint of the study and precluded meaningful statistical analysis. Nonetheless, these results were our first indication that MANP could potentially mitigate MithA-induced toxicity, warranting further refinement of the study design to compare the toxicity profile of free MithA more rigorously and MANP.

FIGS. 9A-9W depict Blood Chemistry for Single Intravenous Injection of 4 mg/kg MithA and MANP in Female C57BL/6J Mice. Blood was collected on day 6 after the initial treatment and prepared for blood chemistry. (9A) Body weight change (%). Statistically significant differences by ANOVA (p≤0.05) indicated by mean body weight change (%) of 1×4 mg/kg MithA vs a=1×4 mg/kg MANP. (9B) Liver/body weight (%) calculated by liver (g)/body weight (g)×100%. Data are expressed as mean±1 SD, n=4 for 1×4 mg/kg MithA, and n=8 for 1×4 mg/kg MANP. Statistically significant differences by t-test (p≤0.05). Representative data for serum levels of (9C) Alanine transaminase. (9D) Aspartate aminotransferase (9E) Alkaline phosphatase. (9F) Total bilirubin. (9G) Glucose. (9H) Blood urea nitrogen. (9I) Serum albumin. (9J) Total serum protein. (9K) Serum globulins. Statistically significant differences by t-test (p≤0.05).

Supplemental FIG. 4.4. (Continued) Representative data for serum levels of (L) Creatinine. (M) Total CO₂. (N) Total Calcium. (O) Chloride. (P) Potassium. (Q) Sodium. Statistically significant differences by t-test (p≤0.05).

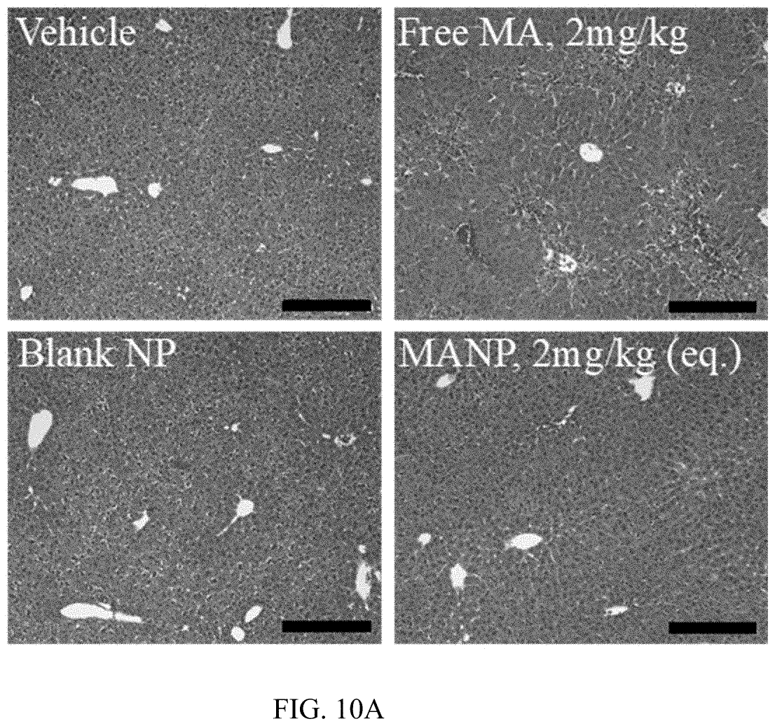

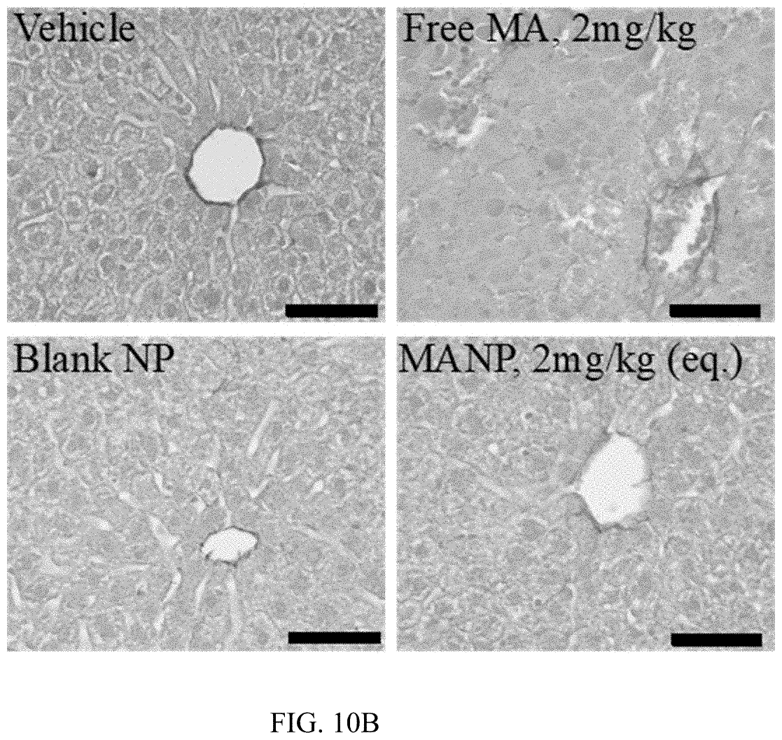

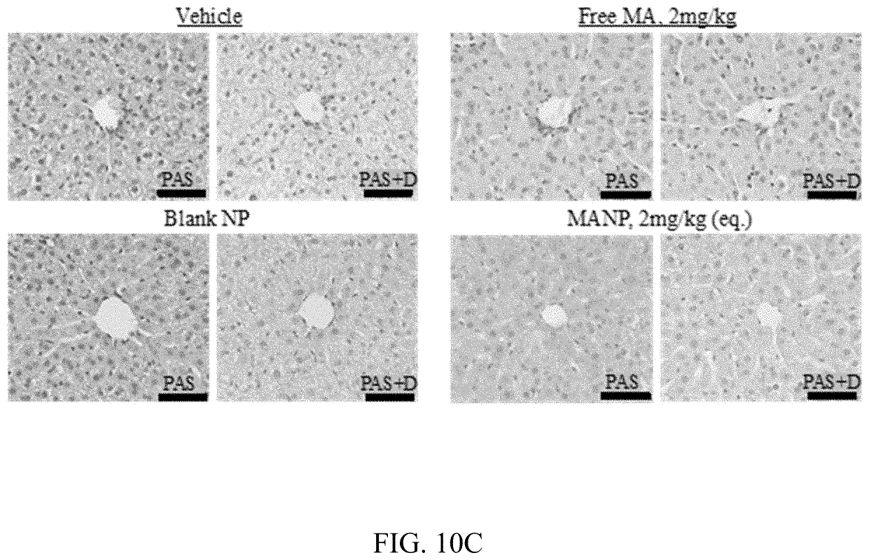

FIGS. 10A-10C depict representative formalin-fixed paraffin sections (5 μm) of liver tissue from mice treated with five doses of saline Vehicle, BlankNP, Free MithA (2 mg/kg) or MANP (2 mg/kg eq) given on alternating days. (10A) Hematoxylin and Eosin stain, 10× magnification; bar shows 200 μm. Free MithA treated mice show periportal hepatocellular necrosis and infiltrates of inflammatory cells, and distension of collecting ducts consistent with acute drug-induced liver injury. These features were not observed in the other treatment groups. FIG. 10B depicts representative formalin-fixed paraffin sections (5 μm) of liver tissue from mice treated with five doses of saline vehicle, BlankNP, Free MithA (2 mg/kg) or MANP (2 mg/kg eq) given on alternating days. (B) Hall's bile stain (olive green) with VanGieson counter stain (yellow/red), 40× magnification; bar shows 50 μm. Non-necrotic hepatocytes in the Free MithA treated group show retention of bile in large vacuoles. in contrast to finely dispersed cytoplasmic bile granules observed in the other groups. These features were not observed in the other treatment groups. Collagen staining was similar between groups.

FIG. 10C depicts Representative formalin-fixed paraffin sections (5 μm) of liver tissue from mice treated with five doses of saline vehicle, BlankNP, Free MithA (2 mg/kg) or MANP (2 mg/kg eq) given on alternating days. (C) Periodic Acid-Schiff (PAS) stained tissue demonstrating glycogen (magenta); hematoxylin counterstain 40× magnification; bar shows 50 μm. Adjacent sections were pre-digested with diastase to remove glycogen prior to staining. The lack of difference between the PAS and PAS+D sections indicates glycogen depletion free MithA treated mice.

Upon observation, that the route of administration could change the toxicity profile dramatically, we revised our experimental design. In the next toxicity experiment, mice were administered five i.v. injections, on alternating days, of free MithA, MANP at 1 mg/kg or 2 mg/kg, or control injections of equivalent volumes of either saline vehicle or blank nanoparticles. All the animals tolerated the treatments under this experimental design, as there were no obvious outward signs of morbidity or toxicity (FIG. 11A). However, ex vivo studies revealed that 5×2 mg/kg MithA group showed the same trend of liver weight reduction as the higher i.p. treated group (5×4 mg/kg MithA) (FIG. 11B and FIG. 7B). Serum chemistry analysis revealed that that 5×2 mg/kg MithA (i.v.) led to 20.9-fold increase in ALT (p<0.0001), 4.1-fold increase in AST (p=0.0023), 1.5-fold increase in total bilirubin (p=0.009), and 1.9-fold decrease in glucose serum level (p=0.0023), compared to saline vehicle group (FIG. 11A-11Q). From these results it is clear that 5×2 mg/kg MithA via i.v. injection led to hepatoxicity in these mice, whereas mice treated with an equivalent dose of MANP did not develop liver toxicity compared to either saline or blank NP (FIG. 11). There was no evidence of kidney injury or electrolyte imbalance in any treatment groups.

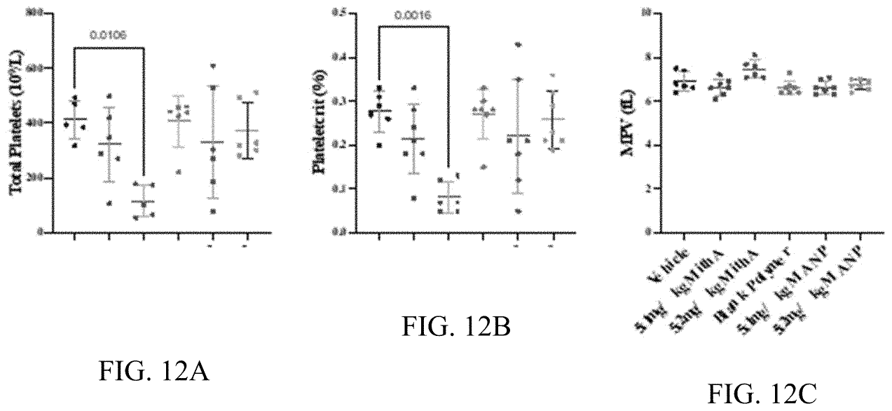

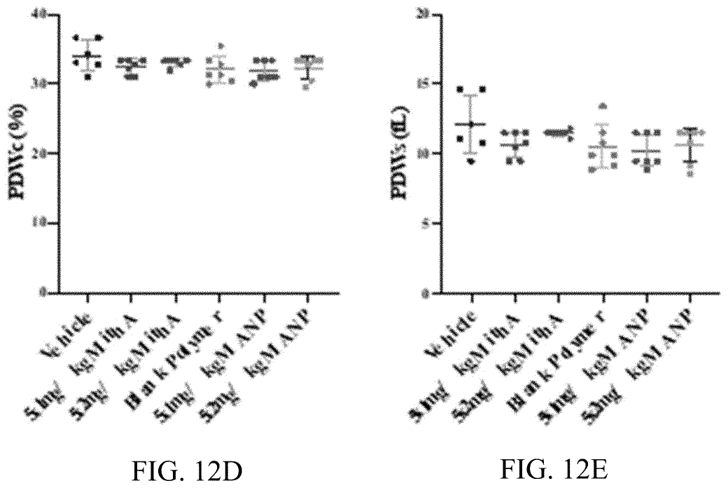

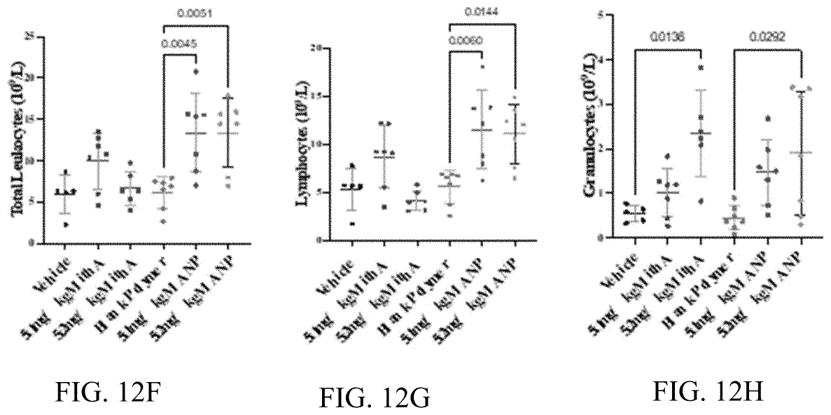

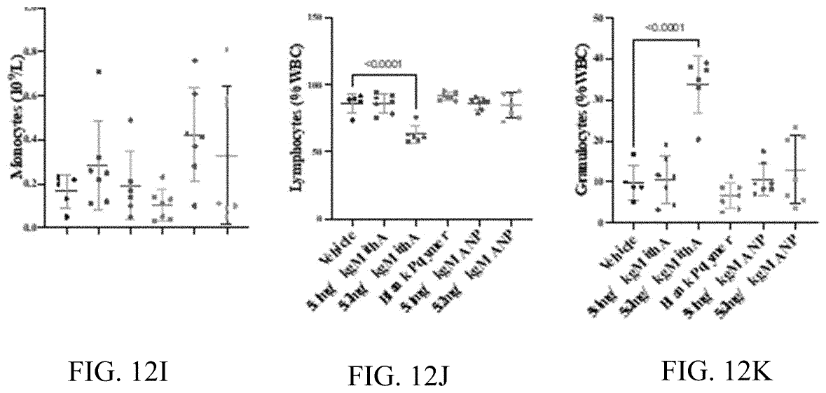

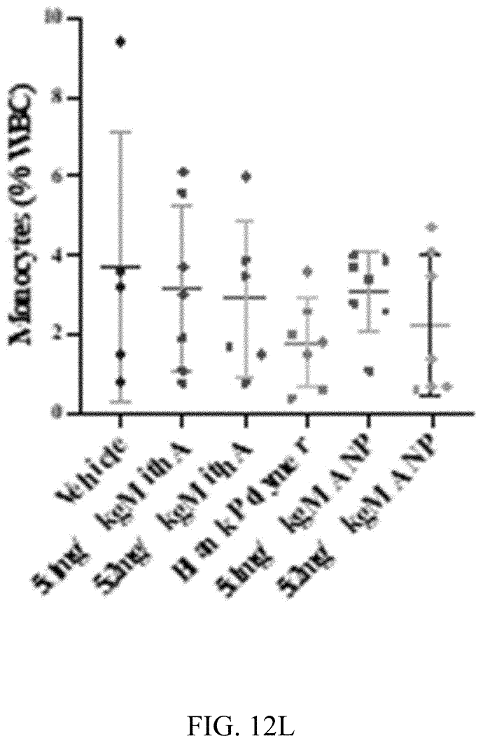

FIGS. 11A-11Q depict MANP Alleviated MithA-induced Liver Toxicity in Female C57BL/6J Mice. Mice were randomized to receive five i.v. injections of saline vehicle, blank polymer, MithA or MANP at corresponding treatment design (see Table S2 for more details) on alternating days. Blood was collected 24 h after the last dose and prepared for blood chemistry. (A) Body weight change (%). (B) Normalized liver weight change (%) calculated by liver (g)/body weight (g)×100%. Data are expressed as mean±1 SD, n=6-9/group. Statistically significant differences by ANOVA (p≤0.05). Representative data for serum levels of (C) Alanine transaminase. (D) Aspartate aminotransferase (E) Alkaline phosphatase. (F) Total bilirubin. (G) Glucose. (H) Blood urea nitrogen. (I) Serum albumin. (J) Total serum protein. (K) Serum globulins. Statistically significant differences by ANOVA (p≤0.05). Representative data for serum levels of (L) Creatinine. (M) Total CO₂. (N) Total Calcium. (O) Chloride. (P) Potassium. (Q) Sodium. Statistically significant differences by ANOVA (p≤0.05). Mice in the 5×2 mg/kg MithA (i.v.) group displayed thrombocytopenia, another well-known MithA-associated toxicity observed in humans. The hematology results reveled a 3.6-fold decrease of total platelet count (p<0.0001), and 3.4-fold decrease of plateletcrit (%) (p=0.0001) in comparison to saline vehicle treated. MithA induced platelet dysfunction was not observed in mice treated with an equivalent dose of MANP (FIG. 12A-12L). No changes in RBC parameters were observed between treatment groups. Surprisingly, we observed an elevation on total WBC counts in MANP treated groups, primarily due to increased total leukocytes (2.2-fold increase), and lymphocytes (2-fold increase) in both 5×1 mg/kg and 5×2 mg/kg MANP treated groups, with p≤0.006 compared to blank polymer group (FIG. 12A-12L). Interestingly 5×mg/kg MithA reduced lymphocytes (% WBC), but increased granulocytes (% WBC), (p<0.0001) which may relate to an inflammatory response hepatoxicity/hepatocellular necrosis compared to saline vehicle group. This possibility was not explored here.

FIG. 12A-12L depict MANP Alleviated MithA-induced Platelet Dysfunction. Representative data for serum levels of (M) Erythrocytes. (N) Hemoglobin. (O) Hematocrit (%). (P) Mean corpuscular volume. (Q) Mean corpuscular hemoglobin. (R) Mean corpuscular hemoglobin concentration. (S) RCWc (%). (T) RCWs. Statistically significant differences by ANOVA (p≤0.05). Representative data for serum levels of (F) Total Leukocytes. (G) Lymphocytes. (H) Granulocytes. (I) Monocytes. (J) Lymphocytes (% WBC). ((K) Granulocytes (% WBC). (L) Monocytes (% WBC). Statistically significant differences by ANOVA (p≤0.05). Representative data for serum levels of (M) Erythrocytes. (N) Hemoglobin. (O) Hematocrit (%). (P) Mean corpuscular volume. (Q) Mean corpuscular hemoglobin. (R) Mean corpuscular hemoglobin concentration. (S) RCWc (%). (T) RCWs. Statistically significant differences by ANOVA (p≤0.05).

### Discussion

In vitro characterization and validation of MANP clearly demonstrated that PEG^{5K}NO₂B was successfully conjugated to MithA, as MANP in PBS did not show a band after running in the mobile phase. Furthermore, it was demonstrated that the When MANP was dissolved in an acidic condition of pH5, free MithA was released from the NPs. This indicated that pH-dependent drug release could further reduce off target injury by restricting the release of the free drug to acidic cellular compartments such as the lysosome, and may also enhance drug permeation by increasing drug lipophilicity and improve drug water solubility.

MANP showed excellent inhibition of EWS tumor cells growth in vitro and in vivo, and suppressed EWS:Fli1 downstream target (NR0B1) and DNA DSB by HRR (Rad 51) at protein level. As expected, the average IC₅₀ was strikingly lower in four cell lines expressing the EWS:Fli1 fusion (10 nM, previous study was 12 nM), compared to Ewing tumor lines lacking this fusion (348.8 nM) and the non-tumor line HEK293T (90.4 nM) for MithA treated 72 hours. Furthermore, MANP treatment significantly extended the survival of TC-71 xenograft tumor bearing mice at doses that did not provoke hepatotoxicity or thrombocytopenia. Moreover, these in vitro and in vivo results are in line with our earlier published findings and concurs well with some other groups demonstrating that MithA is a potent and selective EWS:Fli1 inhibitor. However, the average IC₅₀ values for MANP was approximately 3-fold higher than free MithA for EWS:Fli1⁺ and non-tumor lines (Table 1). This could be due to our nano-formulation is a prodrug form, so it performs less effectively in vitro because it needs to undergo an enzymatic and/or chemical transformation in vivo to release the active free MithA.

HepG2 is a human hepatoblastoma cell line that is widely used to model liver physiology and drug metabolism. Knowing that MithA treatment causes severe hepatotoxicity in humans, it is reasonable that those hepatoblastoma cells are somewhat sensitive to MithA. However, HepG2 was 5.4-fold less sensitive to MANP than MithA, suggesting that our nano formulation may be less toxic to the liver cells than free MithA, and this was supported by our in vivo serum chemistry results (FIG. 4.5).

Historically MithA was used to treat patients with hypercalcemia, Paget's disease of bone and chronic myeloid leukemia, and was believed to act by stopping bone resorption via inhibition of osteoclast activity. THP-1 is a monocytic cell line derived from an acute monocytic leukemia patient, that can be induced to differentiate into osteoclasts using a high-dose of phorbol-12 myristate-13 acetate (PMA) followed by incubation with receptor activator of nuclear factor k-B ligand (RANKL) and macrophage colony-stimulating factor (MCSF). These cells were used for the sensitivity of THP-1 cells to MithA and MANP, and found the IC₅₀ of THP-1 cells was 233.7 nM and 453.6 nM respectively, which may indicate that neither MithA nor MANP were very potent against these model monocytic cells.

Thrombocytopenia is another off-target toxicity observed in humans treated with MithA. The MEG-01 cell line has been used to model megakaryocytes development and platelet production. Interestingly, native Fli1, whose DNA binding domain is preserved in the EWS:Fli1fusion, plays a key role in terminal megakaryocytic maturation and differentiation, and is highly expressed in this cell line. Furthermore, monoallelic deletion of Fli1 is associated with mild thrombocytopenia. Therefore we used Meg-01 as a surrogate to evaluate the effects of MithA on megakaryocyte viability. The average IC₅₀ for MEG-01 cells was 37.8 nM for MithA, and 201.9 nM for MANP (Table 4.1.). These IC₅₀ values suggest that while MithA may have a direct impact on megakaryocytes and that manifests of thrombocytopenia, MANP can greatly reduce these cells. This is further supported by our hematology data which demonstrated that MANP attenuated the MithA-induced platelet defect in vivo.

Biodistribution studies demonstrated MANP mainly accumulated in the tumor and kidney. It is possible that the high renal signal is due to passive retention of nanoparticles in the glomerular filtration apparatus. Given the circumstance that in our biodistribution study, mice were only treated with a single i.v. injection of MANP at 1 mg/kg, we can conclude that the accumulation of MANP in the kidney appears to not result in renal dysfunction because in toxicity study MANP treated groups received a total of 5 doses of 2 mg/kg MANP did not led to elevation of creatinine (marker for kidney function). There was some signal evident in the lungs, but it was only at 4 h time point, which may be due to passive trapping of the MANP and subsequent clearance in the alveolar capillary network. Liver is a well-known barrier for nano drug delivery since, which may similarly trap NPs. However, our MANP do not seem to substantially persist in the liver at later time points (72 h). Lastly, there were very minimal uptake/retention of MANP in the spleen, muscle, brain and heart.

We are aware that our research may have some limitations. The first is that it is possible that at 75% power setting we used on Ami-X system, it may saturate the images that were taken at the earlier time points (1-8 h hours). Thus, one may encounter a lower signal to background ratio. However, it is with understanding that in vivo optical imaging is a qualitative measure of biodistribution because the intensity is not absolutely correlated to the amount of nanoparticles present in the tissues. Secondly, one is unable to detect significant inhibition of NR0B1 and Rad51 after 10 nM MithA treatment at the mRNA level in 3 cell lines tested. However, at lower concentrations (20 nM), it only had a minimal effect on NR0B1. It is likely that mithramycin partially blocks EWS:Fli1-driven transcription initiation, but EWS tumor cells may be capable of recover it by actively transcribe EWS:Fli1 target genes. Thus, with the concentration of 10 nM we did not see inhibition of MithA at EWS:Fli1 targets is not that unreasonable. According to Monument et al, NR0B1 mRNA expression was lowest in tumors with NR0B1 GGAA-microsatellites containing 17-18 GGAA repeats. RD-ES (EWS:Fli1⁺) cells, has 17 X-GGAA-motifs. This might explain why we did not detect NR0B1 expression in RD-ES cells, but we detected NR0B1 in two other EWS:Fli1⁺ cells (A673 and TC-71) containing 24-26 X-GGAA-motif.

The inventors surprisingly found that survival of MANP treated mice was significantly less than that observed for free MithA. However, this finding does conform to an in vitro observation that the MANP were less potent than an equivalent dose of the free drug. Whether difference in potency is due to different kinetics of cellular drug uptake, drug release or metabolism of the MANP pro-drug. The current research did not account for use of MANP at 2 mg/kg dose, which is the dose MithA led to hepatoxicity in mice. There is a high possibility that if one treats tumor-bearing mice with five i.v. injections of 2 mg/kg MANP, one may reach a better anti-tumor efficacy than the free MithA. Since there was toxicity observed in 5×2 mg/kg MANP (i.v.) group, one could potentially use a dose that is even higher than 2 mg/kg to achieve a better local tumor control. However, this cannot be achieved in free MithA group (FIG. 6A-6D). Lastly, in the single i.v. injection of 4 mg/kg MithA and MANP treated mice, a saline vehicle and blank polymer group for control comparisons were not included because of the massive toxicity that warranted cessation of the study for human reasons, Thus, given the small sample size in 4 mg/kg MithA group due to high death rate after injection, one is not able to make conclusive interpretations. However, this study provides the framework for pre-clinal evaluation of MithA in the mouse model. The inventors are the first to report the MithA toxicity in mouse model in this comprehensive matter, including 14 different treatment approaches and two routes of administration.

The present study demonstrates that MANP formulation of the present disclosure retains anti-tumor efficacy while circumventing dose limiting toxicity. Both free MithA and MANP showed excellent potency against EWS:Fli1⁺ cells in vitro. Biodistribution studies demonstrated nanoparticle accumulated in the tumor sites and kidneys, but there was no evident of kidney injury from hematology tests. Animal studies recapitulated the clinical responses of MithA-induced hepatotoxicity and thrombocytopenia observed in human trials. Furthermore, pre-clinical evaluation of MANP anti-tumor activity through an EWS:Fli1 xenograft tumor-bearing mouse model showed that MANP could be a promising anti-tumor agent and is worth further investigation. It is possible further refinement of MANP focused on optimizing either the dosing, schedule could improve the therapeutic efficacy. An alternate strategy could focus on an active targeting NP-based drug delivery strategy such as using an antibody to ES cell-surface molecules such as CD99 to improve MANP retention and internalization.

Most importantly, the MANP formulation of the present disclosure can be administered at higher dose but non-toxic to the liver and alleviated MithA-induced thrombocytopenia. A key barrier for clinical use of MithA against ES is addressing the systemic toxicity of this drug, our pre-clinical xenograft model and toxicity studies demonstrated utilizing nanodrug delivery technique can mitigate the MithA hepatotoxicity. Taken collectively our studies provide a potential effective and safer molecular targeted treatment for ES patients harboring EWS:Fli1 oncogenic fusion.

### APPENDIX

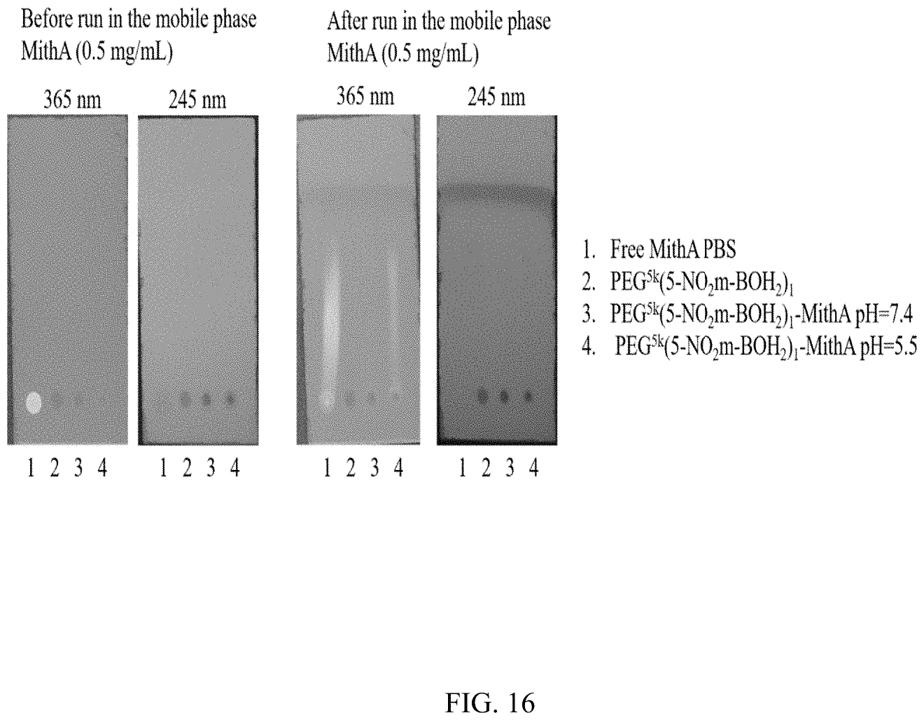

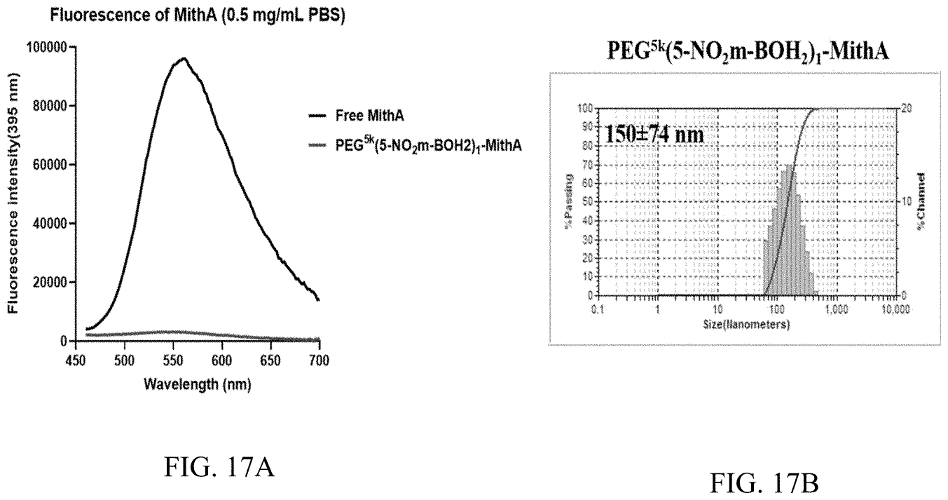

Development and characterization of a nano-formulation Mithramyin A (MANP). A nano-formulation of MithA (MANP) was developed by conjugating PEK^{5K}NO₂B telodendrimeric polymer with a pH-sensitive boronic linkage to MithA (FIGS. 16 and 17 were obtained from Luo's lab). Luo's lab performed the in vitro validation of MANP by thin-layer chromatography to determine the conjugation of MithA to PEG^{5K}NO₂B, and in vitro characterization of MANP drug release at pH 5, and fluorescence reading. Particle size distribution analysis was performed using dynamic light scattering (DLS).

In vitro characterization and validation of PEG5KNO2B—MithA (MANP) conjugation. Luo's lab developed a novel nano-formulation of MithA (MANP) by conjugating PEG^{5K}NO₂B polymer to MithA through a pH-sensitive boronic linkage (FIGS. 13-17). A thin-layer chromatography (TLC) experiment was performed to determine whether PEG^{5K}NO₂B polymer was successfully conjugated to MithA. In TLC, solutes travel with the solvent based on their polarity and acceleration due to gravity. MithA is known to display autofluorescence under UV light at 365 nm (FIG. 17B). PEG^{5K}NO₂B is used as a polymer control (blank polymer). Free MithA traveled with the mobile phase while the blank polymer stayed at the bottom of the TLC as expected. Samples of MANP prepared PBS at pH 7.4 did not show any band in TLC. In contrast, TLC of MANP dissolved in acidic solvent at pH 5 showed release of free MithA, demonstrating that the boronic linkage was indeed pH sensitive. The finding of no autofluorescence in MANP at neutral pH suggests that conjugation to PEG^{5K}NO₂B polymer can the quench auto-fluorescence feature of MithA. To verify this an emission-scanning fluorometry experiment was performed. As shown in FIG. 17A, the fluorescent signal of the free MithA was quenched dramatically after been conjugated to PEG^{5K}NO₂B polymer at a 1:15 mass ratio, in comparison with that of the free MithA. This finding suggests that the UV fluorescence of MithA may be a useful means to assess drug release. Dynamic light scattering indicated the particle size distribution of MANP was 150 nm±74 nm (FIG. 17B.). Taken together, these results indicate PEG^{5K}NO₂B was successfully conjugated to MithA as a prodrug that can be released under low pH conditions.

FIG. 17(A) depicts In vitro validation of MANP by thin-layer chromatography study for drug conjugation and release at pH 5. (B) In vitro characterization of MANP by fluorescence reading and MANP size distribution analysis by DLS. Figure credited to Luo's lab at Pharmacology department of SUNY Upstate Medical University.

### Example 2

Telodendrimer PEG^{5k}(m-BOH₂)ₙ, PEG^{5k}(o-BOH₂)ₙ, PEG^{5k}(p-BOH₂)ₙ, and PEG^{5k}(5-NO₂m-BOH₂)ₙ (n=1 or 2) Preparation. As shown in Scheme 1, telodendrimer comprising n m-, o-, p- or 5-NO₂m-phenylboronic acid moieties was prepared through solution phase peptide synthesis starting from MeO-PEG^{5k}-NH₂.

As shown in Scheme 1(A) (n=1) (FIG. 13), 3-carboxyphenylboronic acid, 4-carboxyphenylboronic acid, 5-carboxyphenylboronic acid, or 3-carboxy-5-nitrophenylboronic acid pinacol ester was conjugated onto the amino group of MeO-PEG^{5k}-NH₂. N,N-Diisopropylethylamine was added to neutralize the hydrochloride on PEG together with N,N′-Diisopropylcarbodiimide (DIC, 3 equiv) and N-hydroxybenzotriazole (HOBt, 3 equiv) added as coupling reagents in dimethylformamide (DMF) to catalyze amide bond formation. The reaction completion was confirmed by the negative Kaiser test result. The ice-chilled ether was added to the reaction solution to precipitate intermediates, and then washed by chilled ether for three times. Then the pinacol ester intermediate was treated by diethanolamine (5 equiv) for 2 h and was precipitated and washed by chilled ether for three times. Subsequently, 0.1 M HCl was added to the intermediate and reacted for 1 h. The final product was directly purified through dialysis against deionized water for 2 days, followed by lyophilization.

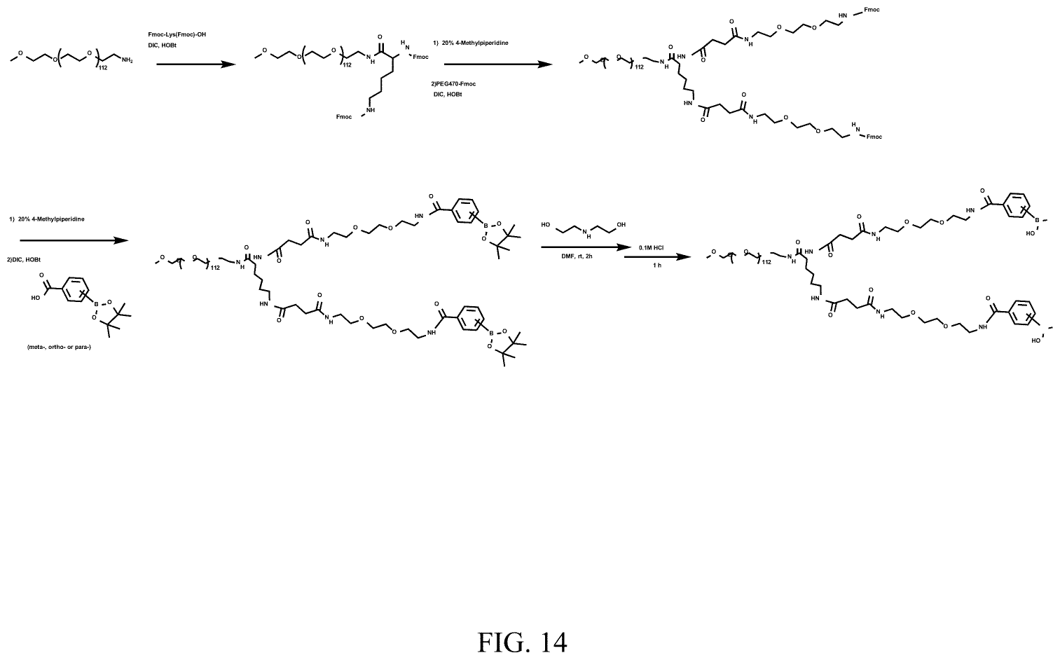

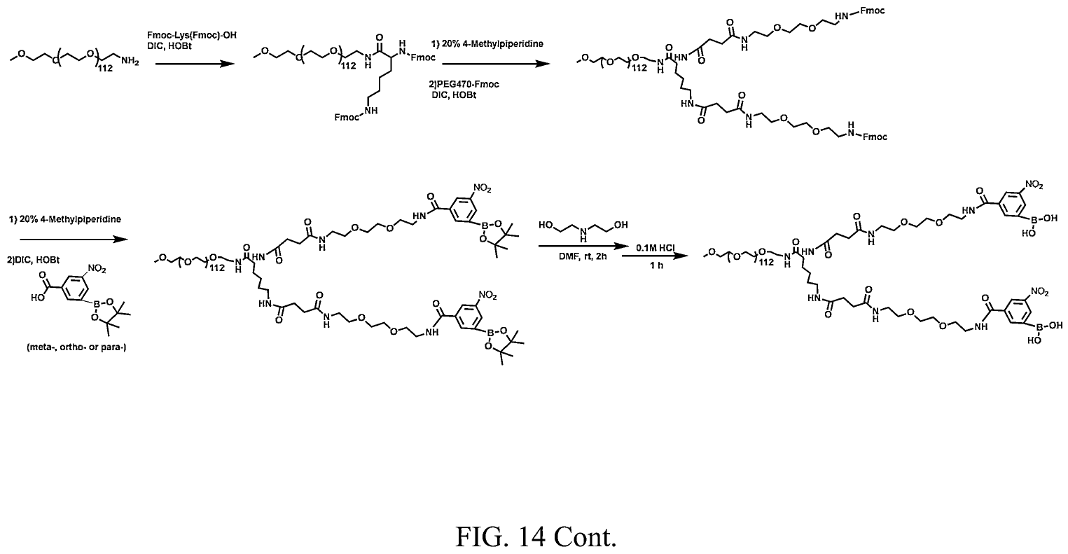

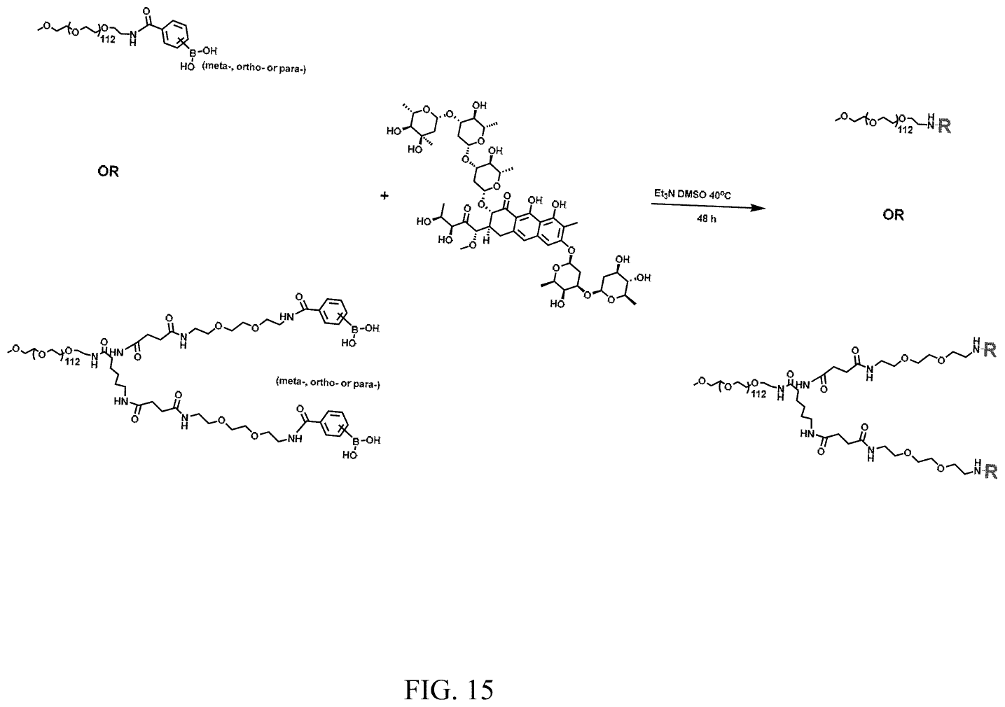

As shown in Scheme 1(B) (n=2) (FIG. 14), Fmoc-Lys(Fmoc)-OH (three equiv) was conjugated onto NH₂ group of PEG^{5k} via coupling reagents HOBt and DIC confirming by negative Kaiser test result. PEGylated materials were precipitated by pouring chilled ethyl ether into reaction solution and washed by chilled ethyl ether twice. Fmoc group was de-protected by 20% (v/v) 4-methylpiperidine in DMF, and the product was precipitated and washed three times by chilled ethyl ether. After vacuum drying at room temperature, PEG⁴⁷⁰-Fmoc linker were coupling on two amino group of lysine. After Fmoc de-protection, 3-carboxyphenylboronic acid, 4-carboxyphenylboronic acid, 5-carboxyphenylboronic acid, or 3-carboxy-5-nitrophenylboronic acid pinacol ester was conjugated on two NH₂ groups. As mentioned above, the precipitation, pinacol ester removal and purification procedures of the products were conducted in the same way.

Preparation of Mithramycin A conjugated Micelles and fluorescence measurement. As shown in Scheme 2, mithramycin A were dissolved in DMSO and was mixed with phenylboronic acid-containing telodendrimers at 1:5 molar ratio. The conjugation was carried out at 40° C. for 48 hours. The reaction completion was confirmed by the TLC detection. After conjugation, Mithramycin A lost its fluorescent under the UV light at 365 nm. After pH 5.5 buffer treatment, mithramycin A in the MithA conjugated micelle formulation exhibited again and migrated similar with free mithramycin, indicating mithramycin A cleavage from telodendrimer nanocarriers at acidic pH. The final product was directly purified through dialysis against deionized water for 2 hours, followed by lyophilization. Fluorescence of mithramycin A and PEG^{5k}(5-NO₂m-BOH₂)ₙ-MithA at 0.5 mg/mL mithramycin A pH 7.4 were detected by BioTeck Synergy H1 with excitation wavelength 395 nm. As shown in FIG. 17B, in comparison with free mithramycin A, the fluorescence of PEG^{5k}(5-NO₂m-BOH₂)ₙ-MithA significantly decreased, indicating successfully conjugation.

Particle size of Mithramycin A conjugated Micelles. Particle size distribution of PEG^{5k}(5-NO₂m-BOH₂)ₙ-MithA was detected by Zetatrac dynamic light scattering (DLS) instrument (Microtrac Inc.) with 10 mg/mL telodendrimer concentration at room temperature. The data was analyzed by volume distribution via Microtrac FLEX Software version 10.6.0. As shown in FIG. 17B, the nanoparticle size of PEG^{5k}(5-NO₂m-BOH₂)ₙ-MithA is 150 nm.

What is claimed is:

1. A nanoparticle compound, comprising:
a hydrophilic polymer, a linker, and a drug, wherein the drug is linked to the hydrophilic polymer by the linker, wherein the drug is mithramycin A or a derivative thereof, and wherein the linker is boronic acid or a boronic acid derivative.

2. The compound of claim 1, wherein the hydrophilic polymer is polyethylene glycol (PEG) having a molecular weight of 1-100 kDa.

3. The compound of claim 1, wherein the boronic acid derivative is selected from the group consisting of 3-carboxyphenylboronic acid, 4-carboxyphenylboronic acid, 5-carboxyphenylboronic acid, 3-carboxy-5-nitrophenylboronic acid pinacol ester, and combinations thereof.

4. The compound of claim 1, wherein the compound is characterized as a PEGylated mithramycin A prodrug, or mithramycin A conjugated micelle.

5. A method of delivering a drug, the method comprising:
administering a nanoparticle compound of claim 1 to a subject in need thereof; and
cleaving the is mithramycin A or a derivative thereof in situ, such that the drug is released from the nanoparticle.

6. The method of claim 5, wherein the subject has a disease characterized as Ewing sarcoma or osteosarcoma.

7. A method of making the nanoparticle compound of claim 1, comprising co contacting phenylboronic acid-containing telodendrimers with mithramycin A under conditions sufficient to form one or more nanoparticle.

8. A method of making a PEGylated mithramycin A prodrug comprising,
contacting one or more phenylboronic acid-containing telodendrimers with a mithramycin A under conditions sufficient to link the one or more phenylboronic acid

9. A nanoparticle comprising: a compound comprising one or more of the following:
(PEG^{5k}(5-NO₂m-BOH₂))₁-MithA;
(PEG^{5k}(5-NO₂m-BOH₂))₂-MithA;
(PEG^{5k}(5-NO₂m-BOH₂))₃-MithA; or combinations thereof.
